(19) United States
(12) Patent Application Publication
(10) Pub. No.: US 2018/0365385 A1
(43) Pub. Date: Dec. 20, 2018

(54) MONITORING AND EVALUATING PATIENT REACTIONS TO MEDICATIONS

(71) Applicant: University Hospitals of Cleveland, Cleveland, OH (US)

(72) Inventors: Matthew M. Cooney, Westlake, OH (US); Patrick M. Mergler, Shaker Heights, OH (US); Saroj Sigdel, North Royalton, OH (US)

(21) Appl. No.: 15/625,697

(22) Filed: Jun. 16, 2017

(51) Int. Cl. G06F 19/00; A61B 5/00

(52) U.S. Cl. CPC G06F 19/3431; A61B 5/4848; G06F 19/322

(57) ABSTRACT

Systems, methods and computer readable media are provided that facilitate monitoring and evaluating patient reactions to medications. In an embodiment, a method can include receiving, by a system comprising a processor, reaction information regarding one or more reactions experienced by a patient in association with usage of a medication by the patient, and generating, by the system, a reaction log for the patient comprising the information. The method can further comprise, providing, by the system, one or more authorized users access to the reaction log, and evaluating, by the system, the reaction information to determine whether the reaction information indicates the patient is experiencing or has experienced one or more clinically significant reactions to the medication.

### TECHNICAL FIELD

This application generally relates to computer processing systems, computer-implemented methods, apparatus and/or computer program products that facilitate monitoring and evaluating patient reactions to medications.

### BACKGROUND

The popular notion is that drugs are thoroughly studied before they are marketed, so that everything about the drug is known at the time of launch. Few realize that no matter how many patients are studied pre-marketing in a controlled environment, the true safety and efficacy profile of a drug is characterized only by continuing safety surveillance through phase IV and post-marketing surveillance (PSM)/non-interventional studies. In particular, the food and drug administration (FDA) approves the efficacy and the common toxicity and/or common side effects information that establish the labeling for the medication based solely on clinical trials. However, this information is often not a good representation of the sample consumer/patients, and is highly controlled. In the clinical setting, the common model of care employed by many medical institutions fails to provide access to phase IV and post market surveillance (PSM) data on drugs. In this regard there isn't systematic follow up in the out-patient setting or even the full clinical setting on what is really happenings to these patients taking these drugs. Accordingly, techniques are needed to improve the quantity and efficiency of ongoing phase IV and PSM studies data on drugs.

### BRIEF DESCRIPTION OF THE DRAWINGS

Numerous aspects, embodiments, objects and advantages of the present invention will be apparent upon consideration of the following detailed description, taken in conjunction with the accompanying drawings, in which like reference characters refer to like parts throughout, and in which:

FIG. 1 illustrates an example system that facilitates monitoring and evaluating patient reactions to medications in accordance with various aspects and embodiments described herein;

FIG. 2 presents an example server medication management component in accordance with various aspects and embodiments described herein;

FIG. 3 presents an example client medication management component in accordance with various aspects and embodiments described herein;

FIGS. 4A-4I presents various graphical user interfaces (GUI)s associated with an application that facilitates monitoring and evaluating patient reactions to medications in accordance with various aspects and embodiments described herein;

FIG. 5 presents an example GUI that facilitates reporting patient reactions to a medication in accordance with various aspects and embodiments described herein.

FIG. 6 provides a high level flow diagram of an example computer-implemented method that facilitates monitoring and evaluating patient reactions to medications in accordance with various aspects and embodiments described herein;

FIG. 7 provides a flow diagram of another example computer-implemented method that facilitates monitoring and evaluating patient reactions to medications in accordance with various aspects and embodiments described herein;

FIG. 8 provides a flow diagram of another example computer-implemented method that facilitates monitoring and evaluating patient reactions to medications in accordance with various aspects and embodiments described herein;

FIG. 9 provides a flow diagram of another example computer-implemented method that facilitates monitoring and evaluating patient reactions to medications in accordance with various aspects and embodiments described herein;

FIG. 10 is a schematic block diagram illustrating a suitable operating environment in accordance with various aspects and embodiments.

FIG. 11 is a schematic block diagram of a sample-computing environment in accordance with various aspects and embodiments.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section.

The subject disclosure is directed to computer processing systems, computer-implemented methods, apparatus and/or computer program products that facilitate monitoring and evaluating patient reactions to medications. In particular, various techniques are provided for automatically receiving feedback regarding reactions to medications take by the respective patients. The feedback can include a variety of forms of clinical information received from various sources that either directly identifies a reaction or that can be correlated to a particular reaction. For example, in one or more embodiments, feedback information regarding a patient's reaction to a medication can include but is not limited to: patient reported reaction data, patient electronic medical record (EMR) data, data collected from a medical monitoring device including wearable devices and implantable medical devices (IMDs) (e.g., physiological data, biomarker data, neurofeedback, etc.), medical imaging data for the patient (e.g., medical images included in imaging studies performed for the patient), laboratory data for the patient, pathology report data for the patient, reaction information reported by a clinician caring for the patient, and the like. The terms “feedback information,” “reaction information,” “patient reaction information,” and the like can any type of information regarding a patient's reaction to a medication, including the various forms of information described above as well other potential types of clinical information that can be used to determine a patient's reactions to a medication. The types of reactions monitored can include side effects of the medication (which can be used to evaluate the medication's toxicity) as well as intended effects of the medication (which can be used to evaluate the medication's efficacy).

In various embodiments, the received feedback information can be collated, cleansed, indexed and provided to the appropriate entities for review such as physicians of the patients, regulatory agencies (e.g., the FDA), and the like (e.g., in the form of a report, or the like). The feedback information can also be regularly and/or automatically (e.g., in real-time or substantially real-time) evaluated by a computing system using one or more algorithms and/or mathematical models to detect parameters and/or patterns in the feedback information indicative of a clinically significant reaction. For example, the computing system can regularly evaluate feedback information regarding a patient's reactions to a medication to determine whether parameters and/or patterns in the patient's reaction information indicate an adjustment to the patient medication regimen is needed. The computing system can further automatically adjust the patient's medication regimen accordingly on an ongoing basis. In some implementations, the computing system can employ one or more implicitly trained or explicitly trained machine-learning techniques to evaluate the feedback information to identify clinically significant reactions. In various embodiments, based on detection of a clinically significant reaction in the reaction information, notifications can be automatically generated and sent to patients, the patients' physicians, pharmaceutical companies manufacturing the medication, regulatory agencies, and the like, identifying the clinically significant reaction. In some implementations, the notifications can also provide a recommended action to perform (e.g., stopping usage of the medication, changing the dosage of the medication, tacking a different medication, contacting emergency services, and the like). In some embodiments, the recommended action can be determined based on evaluation of historical reaction information received for the medication from a plurality of different patients (e.g., thousands to hundreds of thousands or more) using machine learning.

In one or more embodiments, a system is disclosed that comprises a memory that stores computer executable components and a processor that executes computer executable components stored in the memory. The computer executable components can comprise a prompt component configured to generate a prompt at a client device requesting information regarding one or more reactions experienced by a patient in association with usage of a medication by the patient, and a reception component configured to receive first information at the client device regarding the one or more reactions experienced by the patient in response to the generating the prompt. The computer executable component can further comprise a reporting component configured to provide the first information to a server device in response to reception of the first information, wherein based on provision of the first information to the server device, the server device is configured to store the first information in a reaction log for the patient and provide one or more authorized users access to the reaction log.

In various implementations, the prompt can comprise one or more questions regarding the one or more reactions. In some implementations, the one or more questions can be determined based on the medication, a medical health history of the patient, previously reported reactions experienced by the patient, previously reported reaction experienced by other patients, physiological data received for the patient from a medical monitoring device, and the like. In some embodiments, the prompt component can be configured to regularly prompt the patient for reaction information to facilitate monitoring the patient's reactions over time. For example, the prompt component can be configured to prompt the patient in accordance with a schedule (e.g., once an hour, once a day, once a week, once a month, etc.), in response to a trigger (e.g., based on reception of physiological data for the patient indicating the patient may be experiencing a particular reaction to the medication, based on reception of a notification from the server device indicating a new side effect to the medication has been observed, and the like).

In some implementations, the computer executable components can further comprise a notification component configured to receive a notification from the server device in response to a determination, based on the first information, that the patient has experienced or is experiencing a clinically significant reaction. For example, the clinically significant reaction can comprise a side effect and the notification can comprise information instructing the patient regarding a action to perform to stop or minimize the side effect. In another example, the clinically significant reaction can comprise a side effect and the notification can comprise information instructing the patient regarding a change to the patient's medication regimen determined based on the side effect.

In another embodiment, a system is disclosed that comprises a memory that stores computer executable components and a processor that executes computer executable components stored in the memory. The computer executable components can comprise a reaction information reception component configured to receive first information regarding one or more first reactions experienced by a first patient in association with usage of a medication by the first patient. The computer executable components can further comprise a logging component configured to generate a reaction log for the first patient comprising the first information and provide one or more authorized users access to the reaction log, and a patient evaluation component configured to evaluate the first information to determine whether the first information indicates the first patient is experiencing or has experienced one or more clinically significant reactions to the medication.

In some implementations, the computer executable components can further comprise an evaluation response component configured to initiate or execute a response in response to a determination that the first patient is experiencing or has experienced the one or more clinically signification reactions. For example, in one implementation, the response can comprise generating and sending a notification to the patient, the patient's clinician or another entity regarding the one or more clinically significant reactions. In another implementation, the response can comprise changing the patient's medication regimen, including at least one of: revoking the patient's prescription for the medication, changing the patient's prescription dosage for the medication, or prescribing the patient a new medication.

In some embodiments, the reaction information reception component cam further receive second information regarding one or more second reactions experienced by second patients in association with usage of the medication by the second patients, and the logging component can generate historical reaction information for the medication comprising the first reaction information and the second reaction information and provide the one or more authorized users access to the historical reaction information for the medication. According to these embodiments, the computer executable components further comprise a medication evaluation component configured to evaluate the first information and the second information using machine learning to determine whether one or more characteristics associated with patient reactions to the medication that deviate from labeling information developed for the medication relative to a threshold degree of deviation.

In one or more additional embodiments, a method is provided that comprises receiving, by a system comprising a processor, reaction information regarding one or more reactions experienced by a patient in association with usage of a medication by the patient. The method can further comprise generating, by the system, a reaction log for the patient comprising the information and providing, by the system, one or more authorized users access to the reaction log. The method can further comprise evaluating the reaction information by the system to determine whether the reaction information indicates the patient is experiencing or has experienced one or more clinically significant reactions to the medication. In some implementations, in response to a determination that the reaction information indicates the patient is experiencing or has experienced the one or more clinically significant reactions to the medication, the method can further comprise: generating, by the system, a notification regarding the one or more clinically significant reactions, and sending, by the system, the notification to the patient, the patient's clinician or another entity. With these embodiments, the method can further comprise issuing, by the system, an update to the patient's medication regimen, including at least one of: revoking the patient's prescription for the medication, changing the patient's prescription dosage for the medication, or prescribing the patient a new medication.

As used herein, drug can include any substance (other than food) that, when inhaled, injected, smoked, consumed, absorbed via a patch on the skin, or dissolved under the tongue causes a physiological change in the body. In pharmacology, a pharmaceutical drug, also referred to as a medication or medicine, is a chemical substance used to treat, cure, prevent, or diagnose a disease or to promote well-being. The terms medication, medicine, pharmaceutical and therapy agent are used herein interchangeably and respectively refer to a chemical substance used to treat, cure, prevent, or diagnose a disease or to promote well-being.

Various embodiments are described in connection with monitoring and evaluating a patient's reaction to one or more medications. The type of medications can vary and can include both prescription and non-prescription medications. However, it should be appreciated that the disclosed techniques can also be employed to evaluate a user's reactions to substances that are not considered medications (e.g. by the Food and Drug Administration (FDA)). For example, in addition to medications, the disclosed techniques can be used to evaluate a user's reaction to various drugs that are not considered medications, dietary supplements, implantable medical devices (IMDs), surgical implants, biologics (including vaccines, blood and blood products, cellular gene therapy products, and allergenics), cosmetics, tobacco products, foods, food additives, food products and the like. Accordingly, throughout the following disclosure, in various implementations and embodiments, the term medication can be replaced with a non-medication substance (e.g., a drug that is not considered a medication, a dietary supplement, a biologic, a cosmetic, a food, etc.) that is ingested by the patient, applied to the body of the patient, or otherwise introduced into the body of the patient. Information regarding the patient's reaction to the non-medication substance can further be collected, monitored, recorded, evaluated and responded to in a same or similar manner as that described with respect to a medication.

The term patient is used herein to refer to the person that ingests or otherwise has a medication introduced into the body. The term patient generally refers to a person that receives medical treatment. However, it should be appreciated that the term patient can include any person that receives a substance that can have a physiological effect on the body which can be evaluated by the subject systems, including substances that are not considered medications.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

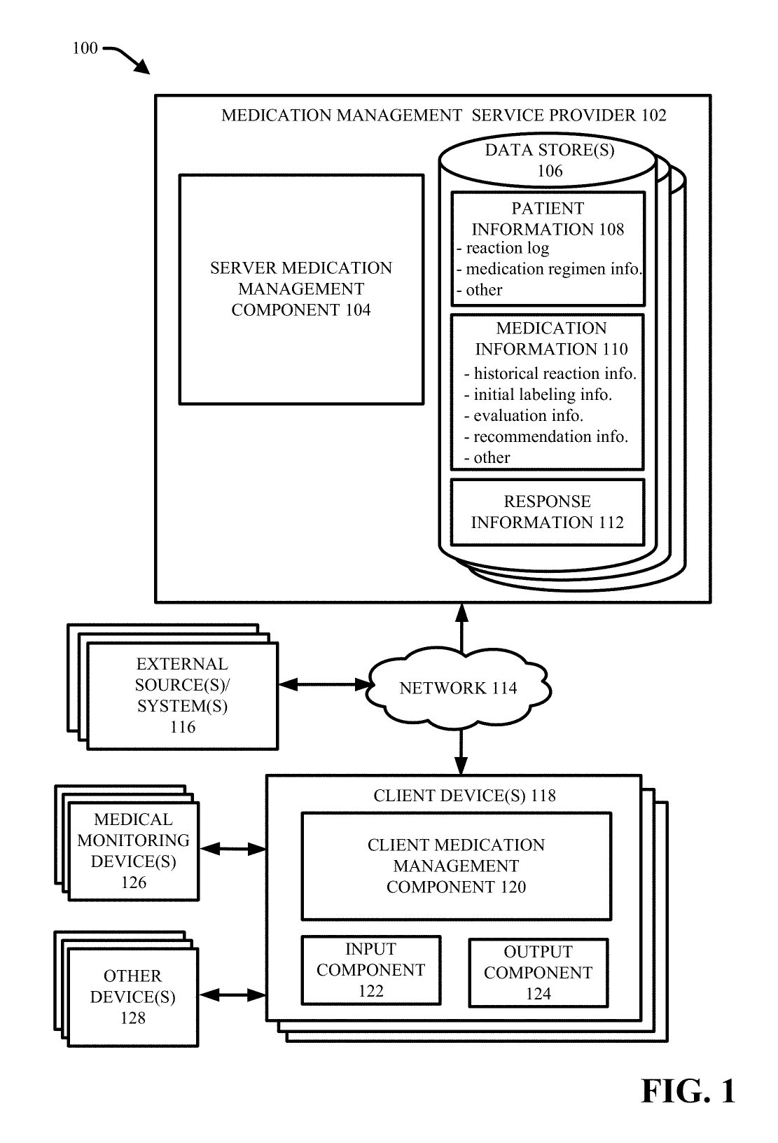

Referring now to the drawings, with reference initially to FIG. 1, presented is a high level block diagram of an example system 100 that facilitates monitoring and evaluating patient reactions to medications in accordance with aspects and embodiments described herein. Aspects of systems, apparatuses or processes explained in this disclosure can constitute machine-executable components embodied within machine(s), e.g. embodied in one or more computer readable mediums (or media) associated with one or more machines. Such components, when executed by the one or more machines, e.g. computer(s), computing device(s), virtual machine(s), etc. can cause the machine(s) to perform the operations described.

System 100 can include a medication management service provider 102 and one or more client devices 118. System 100 can also include one or more external systems/sources 116, one or more medical monitoring devices 126 and/or one or more other devices 128. The medication management service provider 102 can be configured to provide various services associated with monitoring and evaluating patient reactions to medications via a server medication management component 104. In one or more implementations, these services can be embodied in a software application, such as a web-application, a cloud-application, a thin client application, a thick client application, a native client application, a hybrid client application, or the like. For example, in the embodiment shown, the medication management service provider 102 and the one or more client devices 118 can be configured to operate in client/server relationship, wherein the medication management service provider 102 provides the one or more client devices 118 access to information and services provided by the medication management service provider 102 via a network accessible platform (e.g. a web-application, a thin client application, etc.). According to these embodiments, the server medication management component 104 can be configured to perform various back-end processing and reporting functions associated with monitoring and evaluating patient reactions to medications based on reaction information provided by the one or more client device 118. Likewise, the one or more client devices 118 can respectively include a client medication management component 120 configured to perform various front-end processing functions associated with gathering and providing patient reaction information to the server medication management component 104. In this regard, the patient reaction information can include information regarding a patient's reaction to a medication gathered from the patient, or another device associated with the patient (e.g. a medical monitoring device 126 or another device 128), at the client device 118 via a client medication management component 120 provided at the client device 118.

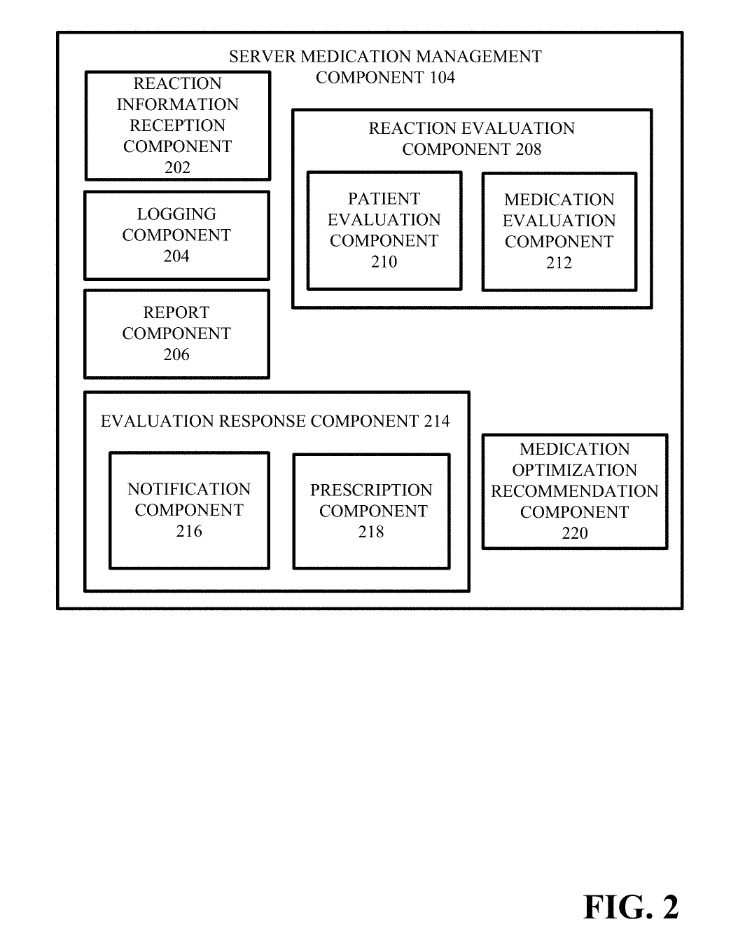

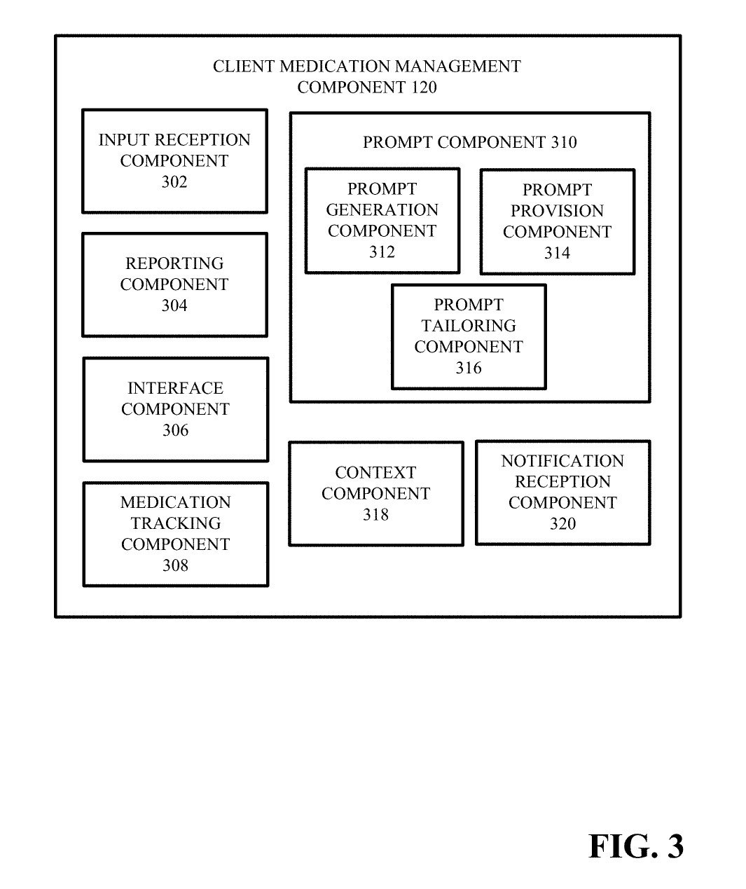

However, it should be appreciated however that system 100 is not limited to this architectural configuration. For example, in some embodiments, one or more components of the medication management service provider 102, including the server medication management component 104, one or more components of the server medication management component 104 (discussed infra with reference to FIG. 2), and information included in the one or more data stores 106, can be provided a the client device 118. In other embodiments, one or more components of the client device 118, including the client medication management component 120, and one or more components of the client medication management component 120 (discussed infra with reference to FIG. 3), can be provided at the medication management service provider 102.

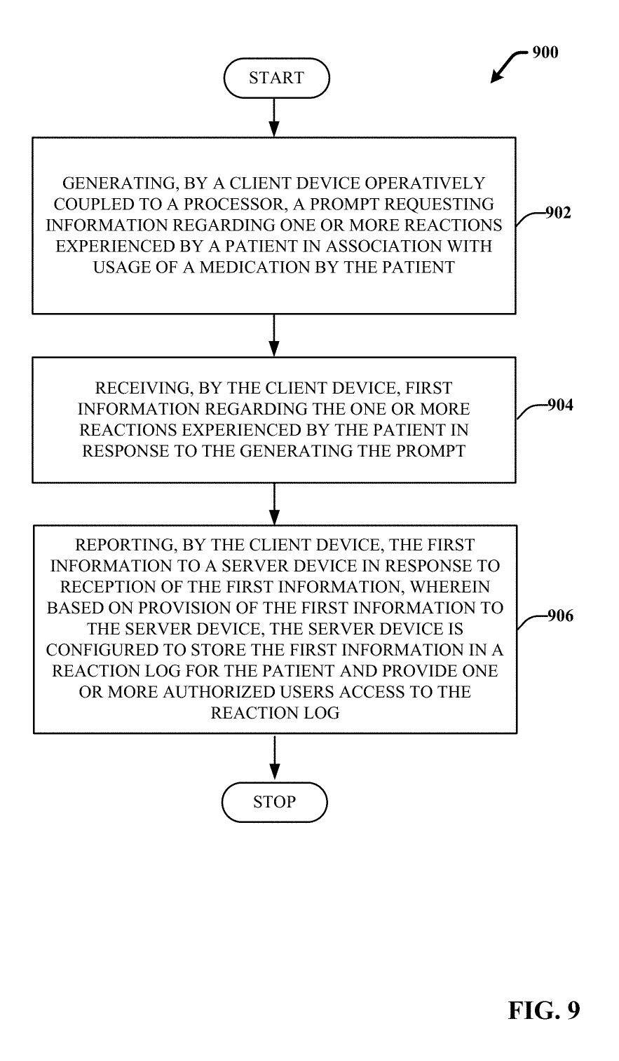

The medication management service provider 102 can include one or more (communicatively coupled) computing devices (e.g. physical devices and/or virtual devices or machines). In this regard, the one or more computing devices of the medication management service provider 102 can include at least one memory that stores executable components (e.g. the server medication management component 104) and at least one processor that executes the executable components stored in the memory. The medication management service provider 102 can also include or otherwise have access to one or more data stores 106 that store and/or provide information (e.g. patient information 108, medication information 110 and response information 112) generated by and/or used by used the server medication management component 104 in association with facilitating the monitoring and evaluation of the patient reaction information. Likewise, the one or more client devices 118 can respectively include at least one memory that stores executable components (e.g. the client medication management component 120), and at least one processor that executes the executable components stored in the memory. Examples of said processors and memories, as well as other suitable computer or computing-based elements that can be included with the medication management service provider 102 and/or the one or more client devices 118, can be found infra with reference to FIG. 9.

The one or more client devices 118 can include any suitable computing device associated with a user and configured to facilitate accessing and employing the information and services provided by the medication management service provider 102. For example, the one or more client devices 118 can include a desktop computer, a laptop computer, a television, an Internet enabled television, a mobile phone, a smartphone, a tablet personal computer (PC), a personal digital assistant PDA, a heads-up display (HUD), virtual reality (VR) headset, augmented reality (AR) headset, or another type of wearable computing device.

In addition to the client medication management component 120, the respective client devices 118 can include suitable hardware and software that facilitates receiving and interpreting user input, displaying or rendering information to the user, and communicating with other devices (e.g. the medication management service provider 102, one or more medical monitoring device 126, one or more other devices 128, the one or more external systems/sources 116, etc.). For example, the respective client devices 118 can include an input component 122 to facilitate receiving and interpreting user input. The input component 122 can include suitable input hardware (and associated software), including but not limited to: a touchscreen, hard or soft buttons, a keyboard, a keypad, a mouse, a joystick, voice/audio input device, a motion/gesture input device, a camera, the like. The respective client devices 118 can also include an output component 124 that includes hardware, software or a combination of hardware and software that provides output information to a user. For example, the output component 124 can include a display screen, a speaker, a vibration apparatus, and the like. The respective client devices 118 can also include suitable communication hardware and associated software (e.g. a central processing unit (CPU), a transmitter, a receiver, a transceiver, a decoder/encoder, etc.) to facilitate wired and/or wireless communication between the client device 118 and other devices (e.g. the medication management service provider 102, the one or more medical monitoring device 126 the one or more other device 128, the external systems/sources 116, and the like).

The various devices, systems/sources, and components of system 100 can be communicatively connected either directly or via one or more networks (e.g. networks 114). Such network(s) can include wired and wireless communication networks, including but not limited to, a cellular network, a wide area network (WAN) (e.g. the Internet), a local area network (LAN), or a personal area network (PAN) or the like. For example, a client device 118 can communicate with the medication management service provider 102, and/or one or more external systems/sources 116 (and vice versa) using virtually any desired wired or wireless communication technology, including but not limited to: a near filed communication (NFC) based protocol, a BLUETOOTH® technology-based protocol, a ZigBee® based protocol, a Wi-Fi protocol, an RF based communication protocol, an IP based communication protocol, a cellular communication protocol, a UWB technology-based protocol, or other forms of communication including both proprietary and non-proprietary communication protocols. In an aspect, one or more devices, systems/sources, and components of system 100 can be configured to interact via disparate networks/communication technologies. For example, in one implementation, a client device 118 can communicate with one or more medical monitoring devices 126 and/or one or more other devices 128 via a LAN or PAN using a close range wireless communication technology (e.g. NFC, BLUETOOTH®, Wi-Fi, etc.), and further communicate with the medication management service provider 102 via a WAN (e.g. the Internet).

The client medication management component 120 can be particularly configured to receive and process input information associated with a patient's reactions to one or more medications take by the patient, referred to herein as patient reaction information, reaction information, and/or feedback information. The feedback information can include a variety of forms of clinical information received from various sources that either directly identifies a reaction or that can be correlated to a particular reaction. For example, in one or more embodiments, feedback information regarding a patient's reaction to a medication can include but is not limited to: patient reported reaction data, patient EMR data, data collected from a medical monitoring device (e.g., medical monitoring devices 126) including wearable devices, IMD and the like, medical imaging data for the patient, laboratory data for the patient, pathology report data for the patient, reaction information reported by a clinician caring for the patient, and the like.

In some embodiments, this patient reaction information can be received over a period of time to facilitate tracking and monitoring of the patient's reactions to a medication taken by the patient over the period of time. For example, the patient reaction information can be collected on a regular basis over the period of time the patient takes the medication and/or or for a defined period of time after the patient stops taking the medication. For instance, in some implementations, the patient reaction information can be collected over this period of time according to a defined schedule (e.g. once an hour, once a day, once a month). In other implementations, the patient reaction information can be collected over this period of time at points when the patient notices or experiences the reaction. According to this embodiment the client medication management component 120 can receive patient reaction information over various periods of time to facilitate evaluating the short term and long term effects of the medication on the patient.

In various embodiments, the patient reaction information can include information regarding side effects the patient is experiencing from the medication. For example, possible side effects can include but are not limited to: attention/memory related side effects (e.g. concentration, memory, etc.); cutaneous related side effects (e.g. acne, hair loss, hand-foot syndrome, hives, itching, pressure sores, nail discoloration, nail loss, nail ridging, radiation skin reaction, rash, sensitivity to sunlight, skin darkening, skin dryness, stretch marks, etc.); cardio/circulatory related side effects (e.g. heart palpitations, swelling, gastro-intestinal, abdominal pain, bloating, constipation, decreased appetite, diarrhea, fecal incontinence, gas, heartburn, hiccups, nausea, taste changes, vomiting, etc.); gynecological/urinary related side effects (e.g. change in unusual urine color, missed menstrual periods, painful urination, urinary frequency, urinary incontinence, urinary urgency, vaginal discharge, vaginal bleeding, vaginal dryness); mood related side effects (e.g. anxious, discouraged, depressed, sad, etc.), neurological related side effects (e.g. dizziness, numbness & tingling, etc.); oral related side effects (e.g. racking at the corners of the mouth or cheilosis, difficulty swallowing, dry mouth, hoarseness, mouth/throat sores, voice quality changes, etc.); pain related side effects (e.g. general pain, headache, joint pain, muscle pain, etc.); respiratory related side effects (e.g. cough, shortness of breath, wheezing, etc.); sexual related side effects (e.g. achieve and maintain erection, desire, ejaculation, pain with sexual intercourse, delayed orgasm, unable to achieve orgasm, etc.); sleep/wake related side effects (e.g. fatigue, insomnia, etc.); visual/perceptual related side effects (e.g. blurred vision, flashing lights, ringing ear, visual floaters, watery eyes, etc.); miscellaneous side effects (e.g. body odor, breast swelling and tenderness, bruising, chills, decreased sweating, hot flashes, increased sweating, nosebleeds, pain and swelling at injection site, etc.). It should be appreciated that the above noted side effects are merely exemplary and can vary depending on the patient, the medication taken by the patient, the dosage of the medication, the frequency the medication is taken, interactions between the medication and other medications the patient is taking, and the like.

The patient reaction information can also include information regarding the efficacy of a medication taken by the patient with respect to the intended purpose of the medication. For example, if the medication is intended to relieve pain, the patient reaction information can include information regarding a level of decrease in the patient's pain. In another example, if a medication is intended to reduce or eliminate a rash, the patient reaction information can include information regarding the degree of reduction of the rash over time.

In various embodiments, information regarding a patient's reaction to a medication can include information received directly from the patient via one or more self-reporting mechanisms provided by the client medication management component 120. For example, the client medication management component 120 can include an application configured to generate or provide a GUI that allows the patient to provide input information identifying and/or describing the patient's reactions to a medication the patient is taking. For example, the GUI can provide for selecting a medication the patient is taking and further provide for identifying and/or describing the patient's reactions to the medication. In some implementations, in order to facilitate receiving patient reaction information, the GUI can include an interactive prompt or form that can be filled out by the patient (or another person that is an authorized agent of the patient). According to these embodiments, the prompt or form can identify one or more defined reactions and allow the user to provide input selecting and/or describing the particular reactions included in the prompt or form that the patient is experiencing. In other implementations, the client medication management component 120 can allow patients to provide input information describing their reactions in free form text (e.g., natural language input).

In addition and/or in the alternative to receiving information regarding patient reactions to a medication via one or more self-reporting techniques facilitated by the client medication management component 120, the client medication management component 120 can receive input regarding the patient's reactions to a medication via one or more medical monitoring devices 126 and/or one or more other devices 128. For example, the one or more medical monitoring devices 126 can include any type of device configured to detect and/or determine physiological information about the patient (e.g. heart rate, blood pressure, SpO₂, respiratory rate, temperature, etc.), and provide the physiological information to the client medication management component 120 (e.g. via a wired or wireless communication signal) in real-time, in response to request, in accordance with a defined schedule (e.g. once an hour, once a day, etc.), or the like.

For example, in some implementations, the one or more medical monitoring devices 126 can include a device that is worn by (e.g. in the form of a wristband, necklace a patch attached to the skin, etc), implanted within (e.g. an implanted medical device (IMD)), or otherwise attached to the patient. With these implementations, the medical monitoring device 126 can be configured to regularly or continuously capture physiological information about the patient over the period of time the medical monitoring device 126 is worn and provide the physiological information to the client medication management component 120 (and/or directly to the server medication management component 104 in some embodiments). In other implementations, the one or more medical monitoring device 126 can include a device that is not attached to the patient that can be operated by the patient or an authorized agent of the patient. For example, the medical monitoring device 126 can include a home device that is intended to remain in the patients' home and be employed by the patient to regularly capture a particular type of physiological information about the patient and provide the physiological information to the client device 118. Further, in some embodiments, the one or more medical monitoring devices 126 can be physically located on or within the client device 118. For example, the client device 118 can include a smartphone (or similar device) configured to detect various physiological measurements using a camera of the smartphone, a pulse oximeter of the smartphone, and/or other types of physiological sensors provided by the smartphone.

The type of physiological information provided by the one or more medical monitoring devices 126 can vary. For example, some implementations, the one or more medical monitoring devices 126 can be configured to detect and/or determine physiological information regarding a patient's heart rate, blood pressure, SpO₂, respiratory rate, temperature, hydration level, glucose level, cortisol level, potassium level, etc. In another example, the one or more medical monitoring devices 126 can include device configured to detect of presence of various bacteria, viruses, pathogens, and the like. In another example, the one or more medical monitoring device 126 can include devices configured to detect presence and/or levels of various biomarkers and/or known substances produced by the body. Other potential information that can be detected by the one or more medical monitoring devices 126 can include but is not limited information regarding a patient's folic acid, calcium, magnesium, creatine kinase, vitamin B12, vitamin D, ferritin, total cholesterol, hemoglobin, HDL, LDL, triglycerides, fatty acids, insulin, hemoglobin, hormones (e.g. thyroid hormones (thyroid-stimulating hormone (TSH), metabolic hormones, reproductive hormones, etc.), liver enzymes, electrolytes (e.g. sodium, potassium, chloride, etc.), platelets, white blood cells, red blood cells, iron, etc.

For example, in some embodiments, the one or more medical monitoring devices 126 can include blood-sampling sensors (e.g. as glucose meters), tissue-embedded sensors (e.g. pacemakers and defibrillators), ingestibles embedded in pills that dissolve, epidermal sensors (e.g. patches and digital tattoos), wearables embedded in clothing or accessories, and external sensors (e.g. blood-pressure cuffs and pulse oximeters). In another example, the one or more medical monitoring device 126 can include a biosensing contact lens configured to be worn in the patient's eye and sense/detect various biomarkers in tear fluid. The contact lens can further be configured to wirelessly transmit information regarding presence and/or concentration of the detected biomarkers to the client device 118. In another example, the one or more medical monitoring devices 126 can an implantable cardioverter-defibrillator (ICD) that is implanted within the patient and configured to perform conventional functions of an ICD as well as additional sensing of biomarkers in bodily fluids. In yet another example, the one or more medical monitoring devices 126 can include a urinary catheter configured to be worn by the user to facilitate urinary functions while also being configured to detect biomarkers in urine and wirelessly transmit information regarding the biomarkers to the client device 118. In another example, the one or more medical monitoring devices 126 can include a device configured to determine physiological information about the user based on detection of one or more biomarkers included in sweat generated by the patient. In yet another example, the one more medical monitoring device 126 can include a blood-testing device that is implanted into the body (e.g. under the skin) or worn in the form of a patch and configured to detect biochemical information regarding at least one of: glucose levels, cortisol levels, potassium levels, blood oxygen levels, blood alcohol levels, inflammation, nitric oxide levels, drug levels/residues present in the body, pathogens present in the body, or bacteria present in the body, folic acid levels, calcium levels, magnesium levels, creatine kinase levels, vitamin B12 levels, vitamin D levels, ferritin levels, total cholesterol levels, hemoglobin, HDL, LDL, triglycerides, fatty acids, insulin, hormones (e.g. thyroid hormones (thyroid-stimulating hormone (TSH), metabolic hormones, reproductive hormones, etc.), liver enzymes, electrolytes (e.g. sodium, potassium, chloride, etc.), platelets, white blood cells, red blood cells, iron, etc.

In another example, the one or more medical monitoring devices 126 can include a device that is configured to detect and determine physiological information regarding electrical activity of the brain (also referred to as neurofeedback) and provide this information to the client medication management component 120. According to this example, the medical monitoring device 126 can include a near-infrared spectroscopy (NIRS) spectroscopy sensor configured to determine haemodynamic information such as working memory performance levels, stress levels and attention levels of the patient. The neurofeedback can also include brain activity information determined by an electroencephalogram (EEG) sensor worn by the patient. For example, EEG measurements can include voltage fluctuations in the time and/or frequency domain that can be correlated to mental performance with respect to various defined cognitive function areas, including but not limited to: attention, familiarity, mental effort, working memory, linguistic acquisition, social learning, and medication.

It should be appreciated that the above described medical monitoring devices 126 (and the associated physiological information capable of being detected/determined by these devices), are merely exemplary and that other existing and future implantable/wearable devices capable of detecting additional types of physiological information can be employed.

The client medication management component 120 can also receive information regarding a patient's reactions to one or more medications from various other devices 128. For example, in one implementations, the one or more other devices 128 can include a scale, pressure pad or another suitable device configured measure information regarding the patient's weight, body fat composition, and/or muscle composition and electronically provide this information to the client device 118. In another implementation, the one or more other devices 128 can include one or more motion sensing devices configured to capture and determine information regarding the patient's range of motion (e.g. using an accelerometer, a gyroscope, a pressure sensor, and the like).

In some implementations, the information (e.g., physiological information, neurofeedback, weight/body composition information, etc.) captured by the one or more medical monitoring devices 126 and/or the one or more other devices 128 can provide a direct measure of a reaction associated with a medication taken by a patient that is either a side effect or an intended effect of the medication. For example, a medical monitoring device 126 can provide a measurement of the patient's temperature indicating the patient has a fever which can be considered a direct side effect of a medication. In another example, a medical monitoring device 126 can provide a measurement of the patient's glucose level indicating that the patient's glucose level has decreased, which can be a direct intended effect of a medication taken by the patient.

In other implementations, the one or more medical monitoring devices 126 can provide physiological feedback that can be correlated (e.g., by the server medication management component 104) to one or more side effects or intended effects of a medication take by a patient. For example, patterns in various combinations of different physiological sensor readings received for a patient at the same time or over a defined period of time can be correlated to one or more known physical or physiological states or conditions which can further be considered an effect of a medication take by the patient. For example, defined patterns or relationships associated with one or more vital sign measurements (e.g. pulse, temperature, blood pressure, respiratory rate, spO₂) received for the patient in combinations and or over a period of time can be correlated to a particular physiological state or condition that can further be considered a reaction to a medication take by the patient. In another example, reception of information from one or more medical monitoring devices 126 and/or other devices 128 indicating the patient has demonstrated low cortisol levels, weight loss, decrease in patient activity levels, etc., which can be correlated to a state of depression, a potential side effect of a medication take by the patient. According to these implementations, the client medication management component 120 or the server medication management component 104 can be configured to process this type of physiological information to determine one or more defined reactions (e.g., side effects or intended effect) to a medication based on known correlations between the physiological information and the defined reactions.

Further, in implementations in which a medication causes a reaction that manifests as one or more visible changes to the patient's body, the client medication management component 120 can also receive image data of the visible changes. For example, a variety of physical reactions to medications can include a visual component, such as but not limited: swelling, a rash, discoloration, moles, cutaneous growths, bumps/lumps, hair loss, hair growth, characteristics of a wound (e.g. healing, infected, etc.). In this regard, the client medication management component 120 can receive image data that depicts any type of visual bodily reaction to a medication. In some implementations, the image data can provide a sequence of images depicting a visual change in appearance over time. For example, the image data can depict an increase or decrease in swelling over time, a change in appearance of a rash over time, and the like. In some implementations, this image data can be captured via a camera of the client device. For example, in implementations in which the client device is a smartphone that includes a camera, the patient (or an authorized agent of the patient) operating the smartphone can take picture of the visual reaction using the camera. In other embodiments, the image data can be captured via another device 128 including a camera and provided to the client medication management component 120.

In various embodiments, the client medication management component 120 can be configured to provide the various input information received regarding a patient's reactions to one or more medications to the server medication management component 104. For example, in some implementations, the client medication management component 120 can be configured to automatically provide the patient reaction information to the server medication management component 104 in real-time or substantially real-time (e.g. as it is received by the client medication management component 120). In other implementations, the client medication management component 120 can be configured to automatically send the patient reaction information to the server medication management component 104 in accordance with a defined schedule (e.g. once a day, once a week, once a month etc.). In another implementation, the client medication management component 120 can be configured to send the patient reaction information to the server medication management component 104 in response to reception of a request from the server medication management component 104 for the patient reaction information. Still in yet another embodiment, the client medication management component 120 can be configured to send the patient reaction information to the server medication management component 104 in response to reception of user input at the client device 118 directing the client medication management component 120 to send the patient reaction information to the server medication management component 104.

In addition to patient reaction information received by the client medication management component 120 from a patient (e.g., via a self-reporting mechanism), from one or more medical monitoring devices 126, from one or more other device 128 and the like, the server medication management component 104 can also receive and/or access various other types of clinical information regarding a patient's reactions to medication. For example, the other types of clinical information can include but are not limited to, patient EMR data, medical imaging data for the patient, laboratory data for the patient, pathology report data for the patient, reaction information reported by a clinician caring for the patient, and the like. In some implementations, this additional patient reaction information can be stored at one or more external sources/systems 116 and accessed by the server medication management component 104 via a network 114. In other embodiments, this additional patient reaction information can be provided to the server medication management component 104 or otherwise retrieved by the server medication management component 104 and stored locally (e.g., at one or more data stores with the patient information 108).

Regardless of the manner in which the server medication management component receives patient reaction information, in some embodiments, the server medication management component 104 can be configured to collate, cleanse and index the patient reaction information. For example, for each patient monitored by system 100, the server medication management component 104 can be configured to generate an indexed, cleansed and organized record including the patient's reaction information. This patient reaction information can be stored in one or more data stores 106, identified in system 100 as patient information 108. The patient's recorded medication reaction information can further be provided to or otherwise made accessible to the patient and/or one or more clinicians involved in the patient's care (e.g., the patients prescribing physician, the patient's pharmacist, etc.) for review and evaluation. For example, in one or more embodiments, the client medication management component 120 can be or include an application that interfaces with the server medication management component 104 and allows authorized users (e.g., the patient, the patient's clinicians, etc.) to access and review various information about the patient, including the patient's medical record which can include the patients medication reaction information. In some implementations, the patient's recorded medication reaction information can further be provided to and/or accessed by one or more external systems/sources 116 including but not limited to, one or more insurance agencies (e.g., the patient's insurance provider), one or more pharmaceutical companies (e.g., the pharmaceutical company that produces the medication), government regulatory agencies (e.g., the FDA), and the like.

The server medication management component 104 can also be configured to generate historical reaction information for each (or one or more) medication evaluated. In particular, the server medication management component 104 can collate, cleanse, organize and index reaction information received from a plurality of patients taking the same medication. This collated medication information can be stored by system 100 in one or more data stores 106. For example, in the embodiment shown, the historical reaction information for respective medications can be stored as medication information 110. In some embodiments, the server medication management component 104 can further be configured to provide the historical reaction information for respective medications to various users and entities, including but not limited to: patient, clinicians, pharmacists, pharmaceutical companies providing the medication, regulatory agencies (e.g., the FDA), and the like. Accordingly, one or more of these entities can employ this information to further improve and regulate the medication prescription requirements, side effects, efficacy, and the like. For example, in some implementation, the server medication management component 104 can automatically provide collated information regarding many (e.g., hundreds to thousands to hundreds of thousands or more) patients' reactions to a medication to the FDA to satisfy post clinical trial regulation requirements required by the FDA. For example, post FDA approval and ongoing regulation/monitoring is generally required by the FDA for most drugs. In this regard, the pharmaceutical companies are required to continue gather data on the effectiveness and side effects of recently marketed medications. However, this is currently an ad-hoc or haphazard process. For example, the current practice many pharmaceutical companies follow to meet this FDA requirement involves merely sending a letter to the physicians prescribing the medication to ask the physicians to report personally observed side effects exhibited by their patients. This process is inefficient, non-scientific, non-data driven, and essentially useless in the grand scheme of actually regulating and understanding the true efficacy and effectiveness of the medication. Accordingly the subject techniques for automatically collecting, collating, indexing and reporting patient reaction information for potentially hundreds of thousands (or more) of patients to regulatory agencies such as the FDA provides a significant improvements in the regard to efficiently collecting and employing patient reaction information in a meaningful way.

In addition to generating the patient information 108 including reported reaction information for respective patients and/or medication information 110 including historical reaction information for respective medications, in various embodiments, the server medication management component 104 can further evaluate the patient reaction information and/or the historical medication reaction information to determine various clinical responses associated with the patient's reactions to the medication and/or the collective patient reactions to the medication.

Various additional features and functionalities of the server medication management component 104 are described infra with reference to FIG. 2, and additional features and functionalities of the client medication management component 120 are described infra with respect to FIGS. 3, 4A-4I and 5.

FIG. 2 presents an example server medication management component 104 in accordance with various aspects and embodiments described herein. In the embodiment shown, the server medication management component 104 can include reaction information reception component 202, logging component 204, report component 206, reaction evaluation component 208, and evaluation response component 214. Repetitive description of like elements employed in respective embodiments is omitted for sake of brevity.

With reference to FIGS. 1 and 2, in various embodiments, the client medication management component 120 can be configured to send the server medication management component 104 the patient reaction information received by the client medication management component 120. This patient reaction information can be received by the reaction information reception component 202. For example, this patient reaction information can include information regarding a patient's reaction to a medication taken by the patient as reported to the client medication management component 120 by the patient via one or more self-reporting mechanisms, the patient's clinician, one or more medical monitoring devices 126 and/or one or more other devices 128. The patient reaction information can also include patient EMR data, medical imaging data for the patient, laboratory data for the patient, pathology report data for the patient, reaction information reported by a clinician caring for the patient, and the like (e.g., received from one or more external systems/sources 116). This additional patient reaction information can also be received and/or retrieved by the reaction information reception component 202 and/or stored locally in the one or more data stores (e.g., as patient information 108).

The logging component 204 can be configured to generate a reaction log for the patient including the patient reaction information. For example, the logging component 204 can collate, cleanse and store the patient reaction information as is it received over time to develop a record (referred to herein as a reaction log) for the patient regarding the patient's reactions to the medication. This patient reaction log can be stored in one or more data stores 106 (e.g., as patient information 108). The server medication management component 104 can further provide (or otherwise make accessible) a patient's reaction log to the patient and/or one or more clinicians involved in the patient's care (e.g., the patients prescribing physician, the patient's pharmacist, etc.) for review and evaluation. For example, in some embodiments, the patient reaction log can accessed by a patient, clinician or authorized user using an application provided by the client medication management component 120 and/or the server medication management component 104 (e.g., a web-application, thin client application, a mobile application, a website, etc.) that allows authorized users (e.g., the patient, the patient's clinicians, etc.) to access and review various information about the patient, including the patient's reaction log.

In other embodiments, the report component 206 can generate a report including a patient's reaction information. For example, the report can include information providing a timeline of the patient's reactions to a particular medication. In some implementations, the report component 206 can distinguish between reactions that are side effects and reactions that are intended reactions of the medication. For example, the report component 206 can generate a report that summarizes a patient's side effects for sequential points in time (e.g., every day, every week, every month, etc.). The report component 206 can also determine information that summarizes a patient's health progress based on the one or more intended reactions of the medication for the sequential points in time. For example, with respect to a medication that is intended to increase hair growth, the report component 206 can generate a report that summarizes the extent of hair growth each week and the side effects experienced by the patient each week. The server medication management component 104 can further provide (or otherwise make accessible) a patient's reaction report to the patient and/or one or more clinicians involved in the patient's care (e.g., the patients prescribing physician, the patient's pharmacist, etc.) for review and evaluation. For example, in some implementations, the report can be accessed by authorized user's using a network accessible platform provided by the client medication management component 120 and/or the server medication management component 104 (e.g., a thin client application, a web-application, a mobile application, a website, etc.). In other implementations, the report component 206 can be configured to send the report to the patient, the patient's physician, or another defined entity (e.g., via an email, a push notification, a fax, or the like).

In some embodiments, the server medication management component 104 can further provide (or otherwise make accessible), a patient's reaction information regarding the patient's reactions to a medication to one or more external systems/sources 116 including but not limited to, one or more insurance agencies (e.g., the patient's insurance provider), one or more pharmaceutical companies (e.g., the pharmaceutical company that produces the medication), government regulatory agencies (e.g., the FDA), and the like. For example, the server medication management component 104 can allow other authorized entities (e.g. the patient's insurance provider, the pharmaceutical company, the FDA, etc.) to access the patient's reaction log using the network accessible platform by the client medication management component 120 and/or the server medication management component 104 (e.g., a web-application, a thin client application, a mobile application, a website, etc.). In other implementations, the report component 206 can be configured to send a report including the patient's medication reaction information to these additional authorized entities (e.g., via an email, a push notification, a fax, or the like).

The logging component 204 can also be configured to can collate, cleanse, organize and index reaction information received from a plurality of patients taking the same medication to generate historical reaction information for respective medications monitored by the medication management service provider 102. This collated information can be stored by system 100 in one or more data stores 106 as medication information 110. For example, the medication information 110 can include historical reaction information monitored for a medication based on a plurality of patients over time. This historical reaction information can provide quantitative and qualitative measurements regarding one or more side effects of the medication and/or efficacy of the medication. This historical reaction information can further relate the respective side effects and efficacy measurements with different parameters associated with dosage and frequency, as well different characteristics of the respective patients (e.g., demographic characteristics, health states/conditions of the respective patients, medical history information associated with the patient, and the like).

In some embodiments, the server medication management component 104 can further be configured to provide the historical reaction information for respective medications to various users and entities, including but not limited to: patient, clinicians, pharmacists, pharmaceutical companies providing the medication, regulatory agencies (e.g., the FDA), and the like. Accordingly, one or more of these entities can employ this information to further improve and regulate the medication prescription requirements, side effects, efficacy, and the like. For example, in some implementation, the server medication management component 104 can automatically provide collated information regarding patient's reactions to a medication to the FDA to satisfy post clinical trial regulation requirements required by the FDA. In one or more embodiments, the historical reaction information for respective medications monitored by the medication management service provider 102 can be sent to one or more of these entities in a summarized report generated by the report component 206. In other embodiments, the historical reaction information generated for respective medications monitored by the server medication management component 104 can be accessed by authorized users via a network accessible platform provided by the server medication management component 104 and/or the client medication management component 120 (e.g., a web-application, a thin client application, a mobile application, a website, etc.).

The server medication management component 104 can also include reaction evaluation component 208 to evaluate the patient reaction information and/or the historical medication reaction information to determine various clinical responses associated with the patient's reactions to the medication. In some embodiments, based on the particular clinical response determined by the reaction evaluation component 208, the evaluation response component 214 can further initiate or otherwise execute the determined response. In the embodiment shown, the reaction evaluation component 208 can include patient evaluation component 210 to monitor and evaluate medication reaction information for individual patients to determine clinical responses associated with the individual patients' care. The reaction evaluation component 208 can also include medication evaluation component 212 to evaluate historical reaction information received for individual medications from many different patients to automatically determine clinical issues, assumptions and associated responses associated with regulating, optimizing and improving clinical applications of the individual medications.

In one or more embodiments, the patient evaluation component 210 can be configured to monitor a patient's medication reaction information over a period of time in which the patient takes the medications and/or after the patient stops taking the medication to evaluate the patient's reaction to the medication and to determine one or more potential clinical responses to perform based on the evaluation. In particular, the patient evaluation component 210 can regularly evaluate medication reaction information received for a particular patient to identify various indicators present in the reaction information indicative of something associated with the patient's health and/or the patient's medication regimen that is considered clinically significant and/or something that should be addressed based on response information 112 included in the one or more data stores 106. For example, the response information 112 can identify specific reactions, values associated with the specific reactions (e.g., regarding intensity, frequency, bodily location, appearance, etc.), patterns in the reaction information, and the like, which have been previously determined to be clinically significant such that if detected, should be responded to with a defined response. Accordingly, the patient evaluation component 210 can regularly compare the received reaction information with the predefined response information to identify notable reactions, values associated with the specific reaction, patterns in the reaction information, and the like, that has been previously associated with a response. In some implementations, the patient evaluation component 210 can monitor and evaluate a patient's reaction information real-time or substantially real-time as the patient reaction information is generated and/or received. In other implementations, the patient evaluation component 210 can be configured to monitor and evaluate the patient's reaction information in accordance with a defined scheduled (e.g., once a day, once a week, once a month, etc.).

The reaction evaluation component 208 can employ various techniques to identify or extract the key values and/or patterns in the patient reaction information depending on the format of the received patient reaction information. For example, in some implementations, the received user reaction information can include direct computer readable values/parameters that can be compared to the defined reaction parameters included in the response information 112. For example, these implementations can include reaction information received in response to user input selecting one or more reactions and/or reaction values in association with filling out a form or responding to a questionnaire that provides for selecting one or more defined reactions/values included in a fixed set of reactions and/or values for those reactions. These implementations can also include reception of a specific parameter from a defined type of medical monitoring device 126 or other device 128 configured to provide a finite set of possible parameters. In other implementations in which the reaction information includes free form text describing a patient's reaction(s) to a medication, the reaction evaluation component 208 can employ one or more natural language processing (NLP) functions to convert the text into computer readable values and/or terms to facilitate identifying specific reactions, reaction values, combinations of reactions, patterns in the reaction information, and the like, which are associated with one or more predefined clinical responses included in the response information 112. Further, in implementations in which the reaction information includes image data providing a visual representation of a patient's reaction, the reaction evaluation component 208 can employ one or more image analysis functions that involve automatically identifying visual features included in the images to detect objects included in the images and associated characteristics of the objects (e.g., color, texture, size, etc.).

The response information 112 can further identify the one or more particular responses to perform in association with recognition of a defined reaction, reaction value and/or pattern in the reaction/value information. For example, the responses can include but are not limited to: instructing the patient to stop taking the medication, instructing the patient to change the dosage/frequency of the medication take, instructing the instructing the patient to perform one or more actions to alleviate an undesired reaction (e.g., rest, elevation, icing, etc.), instructing the patient to contact the patient's physician regarding the reaction, changing the patient's medication, changing the patient's medication dosage/regimen, prescribing one or more additional medications for the patient, notifying the patient's physician regarding the patient's reaction, contacting emergency services, and/or various other possible responses that have been determined clinically appropriate responses given the patient's condition, the medication or medications taken by the patient, and the particular reaction's experienced by the patient.

The evaluation response component 214 can further initiate or otherwise execute the determined response. For example, the evaluation response component 214 can include notification component 216 to generate and send notifications to patients, clinicians or other suitable entities regarding reception of clinically significant or otherwise actionable patient reaction information. In some implementations, the notification can include information identifying the clinically significant or actionable patient reaction information. For example, in implementations in which the patient evaluation component 210 determines a response that includes informing the patient's physician regarding the patient's reactions (as defined by the response information 112), the notification component 216 can generate and send the notification to the patient's physician. The notification can include information identifying the specific reaction the patient is experiencing that should be evaluated by the physician. For example, a notification sent to a patient's physician might include information stating “your patient John Doe has been experiencing frequent dizzy spells and shortness of breath within hours of taking medication abc.”

In other implementations, the response information 112 associated with an identified clinically significant or otherwise actionable patient reaction information can include instructions for the patient, the patient's physician, or another suitable entity, for responding to the reaction information. According to these implementations, if the patient evaluation component 210 determines a response that involves instructing the patient (or another suitable entity) to perform an action (e.g., stop taking the medication, change the dosage of the medication, contact the patient's clinician, contact emergency services, get some rest, stop eating a particular food, etc.), the notification component 216 can generate and send a notification to the patient (or the other suitable entity) including the instruction. For example, the response information 112 can include information that indicates the patient should stop taking a medication and contact the patient's clinician if the patient experiences a particular side effect. According to this example, in response to a determination that the patient experiences the particular side effect, the notification component 216 can send a notification to the patient instructing the patient to stop taking the medication and contact the patient's physician. In another example, a notification sent to a patient in association with received reaction information indicating a particular medication has been causing the patient insomnia might include information stating “in order to improve your sleep, take your medication before 10 am.”

The manner in which the notification component 216 sends a notification to a patient, the patient's physician, or another suitable entity (e.g., the patient's pharmacy/pharmacist, the patient's insurance provider, a medical organization responsible for the patient, a regulatory agency, etc.) can vary. For example, the notification can include but is not limited to: an electronic notification sent to a user's device, a notification sent to a user account accessible via an application or network accessible platform associated with the client medication management component 120, an email notification, a push notification, a text message notification, and the like.

In some implementations, a response associated with a particular reaction, reaction value, and/or pattern in the reaction information can include changing the patient's medication regimen. For example, the response can include revoking the patient's prescription to a particular medication, changing the medication dosage, changing the medication frequency, and/or prescribing the patient another medication. According to these implementations, the prescription component 218 can be configured to automatically change the patient's prescribed medication regimen on behalf of the patient's prescribing physician. In this regard, the prescription component 218 can automatically authorize and issue changes to a patient's prescribed medication(s). For example, the prescription component 218 can automatically notify the patient's pharmacy/pharmacist regarding changes to the patient's prescriptions, including prescribing new medication, changing dosage, revoking a prior prescription, and the like. In some implementation, the prescription component 218 can interface with an electronic pharmacy system (e.g., which can be included at an external system/sources 116) and automatically update the patient's records to reflect the patient's changes to his or her prescriptions. The prescription component 218 can also notify the patient and/or the patient's physician (or another suitable entity) regarding the changes to the patient's prescriptions.

For example, based on evaluation of a patient's reaction information in view of the response information 112, the patient evaluation component 210 can determine that a patient's has experienced side effects of joint pain exceeding a threshold pain level and/or duration in association with usage of a first medication, and that the patient should be prescribed a second medication that is designed to relieve excessive joint pain. The prescription component 218 can further automatically prescribe the patient the second medication by notifying the patient's pharmacy and/or electronic pharmacy system regarding the prescribed second medication for the patient. The prescription component 218 can further send the patient a notification informing the patient that a new medication has been prescribed for the patient that is intended to alleviate the patient's joint pain. The patient can then make a visit to the patient's pharmacy to pick up the medication when it is filled. In another implementation, the patient can automatically receive the second medication in the mail.

In some embodiments, the prescription component 218 can regulate automatic prescription refills based on evaluation of a patient's reaction information. For example, in current clinical settings, for high cost medications, there is often a requirement that a pharmacist call the patient once a month to evaluate how the patient is doing, discuss side effects, discuss medical improvement, and the like. This is a prerequisite for the pharmacist to authorize refilling the prescription for the following month. This is a procedure that is a costly and inefficient process. In some embodiments, the prescription component can eliminate or minimize the costs associated with this process by evaluating patient reaction information and automatically regulating the refill process. For example, in various embodiments, the prescription component 218 can compare the patient's reaction/side effects to a medication over a period of time with standard clinical reaction requirements that must be met in order for the patient to receive a prescription refill each consecutive period of time. According to these embodiments, if the standard clinical requirements are met (e.g., the patient is experiencing no or acceptable side effects and/or the patient is experiencing an acceptable degree of clinical benefit from the medication), the prescription component 218 can automatically authorize the refill with the patient's pharmacist/pharmacy. On the other hand, if the standard clinical requirements are not met, the prescription component 218 can refuse to authorize the refill. In some implementations, in association with a refusal to authorize the refill, the prescription component 218 can further be configured to notify the patient and/or the patient's clinician regarding the refusal to automatically refill the prescription. For example, the prescription component 218 can direct the notification component 216 to send the patient's clinician a message with information indicating the patient's prescription cannot be automatically filled. The message can also include information identifying the factors/problems which prevented the auto refill. The patient's clinician can then further review the patient's reaction information to either override the refill rejection and/or initiate a different clinical approach to the patient's reactions.

In some embodiments, in addition to evaluating side effects experienced by the patient, the patient evaluation component 210 can also be configured to evaluate the patient's reaction information to determine if the medication is improving the patient's health. In this regard, the patient reaction information can include one or more reactions experienced by the patient are intended reactions of the medication. According to these embodiments, the patient evaluation component 210 can access and employ information (e.g., included in patient information 108) regarding the patient's initial health state prior to taking the medication and compare the patient's reaction information over time demonstrating progress (or no progress) in the patient's health state to determine whether the patient's health state is improving and the degree to which the medication is improving the patient's health state is improving. The patient evaluation component 210 can further compare information regarding the degree to which the patient's health condition is improving with predefined response information 112 that provides conditions for responding to the patient's reaction information. For example, the response information 112 can identify responses to perform (e.g., notifying the patient, the patient's clinician, changing the patient's medication regimen, etc.) based on reception of reaction information that indicates the patient's clinical improvement is below or above a threshold requirement. The evaluation response component 214 can further generate and execute the appropriate response (e.g., sending a notification via notification component 216 to the patient, the patient's clinician, or another suitable entity regarding the patient's reaction, changing the patient's prescription via prescription component 218, issuing emergency services, etc.).

In some embodiments, patient evaluation component 210 can tailor the evaluation of a patient's reaction information to determine whether a clinical response is warranted and if so, the particular clinical response, based on personalized aspects associated with the particular patient. For example, in addition to the defined response information 112, the patient evaluation component 210 can evaluate a patient's reaction information based on one or more additional factors, including but not limited to, information regarding a medication regimen of the patient (e.g., including other medications taken by the patient, dosage, frequency, etc.), the current health state/condition of the patient, health history information for the patient, demographic information, previously reported reactions experienced by the patient, laboratory study information received for the patient, imaging study information received for the patient, and the like. This additional information can be included with the patient information 108. Alternatively, the server medication management component 104 can access such additional information for the patient at one or more external systems and/or sources 116.

The medication evaluation component 212 can be configured to evaluate historical reaction information received for individual medications to automatically determine clinical issues, assumptions and associated responses associated with the individual medications. In particular, the medication evaluation component 212 can employ various machine learning techniques to identify patterns in the historical reaction information to determine additional and/or improved clinical information about the reactions caused by a particular medication relative to those determined in association with clinical trials of the medication. For example, the medication evaluation component 212 can identify side effects of the medication, information regarding frequencies of the side effects (e.g., common side effects), characteristics about the side effects (e.g., severity, frequency, location, timing of occurrence, etc.) and the like. The medication evaluation component 212 can also compare this newly developed information with the initial labeling information for the medication to determine whether and how to update the labeling information to include more accurate information regarding the medication side effects. For example, the medication evaluation component 212 can identify new side effects and/or reactions that were not reported following initial clinical trials of the medication. The medication evaluation component 212 can also determine additional and/or more accurate information (e.g., relative to that determined based on clinical trials) regarding efficacy of the medication in association with its intended application, and the like.

Using machine learning, the medication evaluation component 212 can further determine correlations between medication reactions and one or more correlated metrics associated with the patients reporting the reactions and/or the dosage/frequency of the medication taken. For example, the medication evaluation component 212 can determine correlations between certain reactions factors including but not limited to: demographic characteristics of the patients (e.g., gender, age, ethnicity, etc.), health states/conditions of the patients, health history information of the patients, one or more other medications the respective patients are tacking (e.g., interactions between two or more medications), dosage, frequency, medication application method (e.g., orally, injection, topically, etc.), and the like.

In some embodiments, in addition to making various inferences and determinations regarding reactions caused by certain medications and one or more additional factors associated with the patient, cross-medication interactions, dosage/frequency, etc., correlated to certain reactions, the server medication management component 104 can further include medication optimization recommendation component 220 to determine clinical recommendations regarding optimizing application or usage or a particular medication. In this regard, medication optimization recommendation component 220 can employ one or more machine learning techniques to evaluate the positive, negative and neutral reactions associated with taking a medication by a particular type of patient (e.g., based on the patient's demographics, health condition, health history, etc.), at a particular dosage/frequency, and/or in conjunction with one or more other medications. The medication optimization recommendation component 220 can further determine characteristics (e.g., demographic characteristics, medical condition characteristics, medical history characteristics, cross-medication reaction characteristics) of patients that should and shouldn't take the medication based on side effects they are likely to experience and whether the drug will be effective for the particular patient. The medication optimization recommendation component 220 can further determine optimal dosage and frequency prescription recommendations for a particular medication based on efficacy and side effect information received for the medication at different dosages and frequencies.

In one or more embodiments, the server medication management component 104 can store information regarding the various determinations and inferences made by the medication evaluation component 212 and/or the medication optimization recommendation component 220 with the medication information 110. The server medication management component 104 can further provide authorized users (e.g., patients, medical personnel, administrative personnel, medical organizations, pharmaceutical companies, regulatory agencies, etc., access to the medication information 110 (e.g., via a network accessible platform). In other implementations, the report component 206 can include this type of medication information determined by the medication evaluation component 212 and/or the medication optimization recommendation component 220 in a report generated by the report component 206. The report component 206 can further send the report, or otherwise make the report accessible, to authorized users.

In some embodiments, the evaluation response component 214 can also be configured to automatically initiate and/or execute responses based on one or more inferences and/or determinations made by the medication evaluation component 212 and/or the medication optimization recommendation component 220 regarding a particular medication. With these embodiments, the response information 112 can also include information identifying particular determinations and/or inferences that could be determined by the medication evaluation component 212 and/or the medication optimization recommendation component 220 that are considered clinically significant or alarming and thus should be responded to with an automatic notification or other type of response by the evaluation response component 214. For example, in some implementations, the response information 112 can direct the notification component 216 to generate and send a notification to one or more defined entities (e.g., the patient, the patient's clinician, the pharmaceutical company that makes the medication, the FDA, etc.) based on identification of reaction information for a medication that was not observed in clinical trials, such as but not limited to: a new side effect, a side effect that is particularly associated with a newly identified patient characteristic, (e.g., women over the age of 50), identification of a side effect at significantly higher rate than that identified in clinical trials, identification of side effect associated with multi-drug interactions, and the like. In another implementation, the response information 112 can direct the notification component 216 to generate and send a notification to one or more defined entities (e.g., the patient, the patient's clinician, the pharmaceutical company that makes the medication, the FDA, etc.) based on identification of a specific reaction, reaction value and/or pattern in reaction information considered critical or noteworthy (e.g., N or more patient deaths, N or more patients experiencing strokes, N or more patients experiencing relapse, N or more patients experiencing significant recovery/improvement, etc.). In another implementation, the response information 112 can direct the notification component 216 to generate and send a notification to one or more defined entities (e.g., the patient, the patient's clinician, the pharmaceutical company that makes the medication, the FDA, etc.) based on identification of a significant (with respect to a defined threshold) contradiction to original medication labeling information.

In some embodiments, the notification component 216 can be configured to generate and send a notification to the client medication management component 120 in response to determination of new or improved clinically signification reaction information. For example, in response to determining, by the medication evaluation component 212, a new reaction, a new reaction value, a new pattern in reaction information, that is clinically significant based on evaluation of reaction information for the medication received from a plurality of patients, the notification component 216 can be configured to notify the client medication management component 120. Such a notification is referred to herein as a medication reaction update notification. Based on reception of a medication reaction update notification, the client medication management component 120 can be configured to request information from a patient taking the medication (or who have recently stopped taking the medication) regarding whether the patient has experienced the newly identified clinically signification reaction. For example, the client medication management component 120 can be configured to generate and present the patient with a prompt asking the patient if he or she has experienced the newly identified clinically significant reaction. In another example, the client medication management component 120 can be configured to add one or more additional questions to a form or questionnaire about reactions to the medication regarding the newly identified reaction. For example, in response to a determination by the medication evaluation component 212 that a significant number of patients have been experiencing blurred vision in association with a particular medication, the notification component 216 can send a medication reaction update notification to the client medication management component 120 of a client device employed by a patient taking the medication regarding the newly identified side effect. Based on reception of the medication reaction update notification, the client medication management component 120 can then generate a prompt asking the patient (who has not previously reported blurred vision) whether he or she has in fact experienced blurred vision.

The notification component 216 can also be configured to send a notification to a particular patient type/group regarding a newly identified or noteworthy characteristic about a medication determined by the medication evaluation component 212 and/or the medication optimization that is relevant to that patient type group. For example, in response to a determination by the medication evaluation component 212 that a significant number (e.g., relative to a defined threshold) of pregnant women have shown a critical side effect when taking drug xyz, the notification component 216 can be configured to generate and send a notification to only the pregnant women taking drug xyz. The notification can instruct the patients to stop taking the drug immediately and contact their physician.

In one or more embodiments, newly identified clinically significant reaction information for a medication determined by the medication evaluation component 212 can be added to the response information 112. For example, newly identified reactions, reaction values, patterns in the reaction information (e.g., regarding combinations of reactions, correlations between a reaction or reaction value and one or more patient factors and/or dosage factors) and the like, that are considered clinically significant can be added to the response information 112 and associated with a defined response (e.g., sending a notification to the patient, the patient's clinician, or another suitable entity). The patient evaluation component 210 can further employ the updated reaction information when evaluating individual patient reaction information. For example, in response to a newly identified side effect that has been found clinically significant in female patients over 50, the response information 112 can be updated to include information indicating identification of this reaction in female patients over 50 should be responded to with a notification to the patient's clinician. Further, in implementation in which the medication optimization recommendation component 220 determines a optimal response to a particular clinically significant reaction (e.g., changing the medication dosage, stopping the medication and contacting the patient's physician, etc.), this response can associated with the clinically signification reaction in the response information 112 and applied by the evaluation response component 214.

Machine learning is a type of artificial intelligence (AI) that provides computers with the ability to learn without being explicitly programmed. Machine learning focuses on the development of computer programs (e.g., the client medication management component 120 and/or the server medication management component 104) that can change when exposed to new data. Machine learning techniques use that compiled data to detect patterns in the data and adjust program actions accordingly. The machine learning algorithms employed by the medication evaluation component 212 and the medication optimization recommendation component 220 can include supervised or unsupervised algorithms. Supervised algorithms can apply what has been learned in the past to new data. Unsupervised algorithms can draw inferences from datasets. Deep learning is an aspect of AI that is concerned with emulating the learning approach that human beings use to gain certain types of knowledge. At its simplest, deep learning can be thought of as a way to automate predictive analytics. While traditional machine learning algorithms are linear, deep learning algorithms are stacked in a hierarchy of increasing complexity and abstraction. Each algorithm in the hierarchy applies a non-linear transformation on its input and uses what it learns to create a statistical model as output. Iterations continue until the output has reached an acceptable level of accuracy. The number of processing layers through which data must pass is what inspired the label “dee” p.

In order to provide for or aid in the numerous inferences described herein, the server medication management component 104 can examine the entirety or a subset of the data to which it is granted access and can provide for reasoning about or infer states of the system (e.g., system 100 and the like), environment, etc. from a set of observations as captured via events and/or data. An inference can be employed to identify a specific context or action, or can generate a probability distribution over states, for example. The inference can be probabilistic (e.g., the computation of a probability distribution over states of interest can be based on a consideration of data and events). An inference can also refer to techniques employed for composing higher-level events from a set of events and/or data.

Such an inference can result in the construction of new events or actions from a set of observed events and/or stored event data, whether or not the events are correlated in close temporal proximity, and whether the events and data come from one or several event and data sources. Various classification (explicitly and/or implicitly trained) schemes and/or systems (e.g., support vector machines, neural networks, expert systems, Bayesian belief networks, fuzzy logic, data fusion engines, etc.) can be employed in connection with performing automatic and/or inferred action in connection with the claimed subject matter.

A classifier can map an input attribute vector, x=(x1, x2, x4, x4, xn), to a confidence that the input belongs to a class, such as by f(x)=confidence(class). Such classification can employ a probabilistic and/or statistical-based analysis (e.g., factoring into the analysis utilities and costs) to prognose or infer an action that a user desires to be automatically performed. A support vector machine (SVM) is an example of a classifier that can be employed. The SVM operates by finding a hyper-surface in the space of possible inputs, where the hyper-surface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, and probabilistic classification models providing different patterns of independence can be employed. Classification as used herein also is inclusive of statistical regression that is utilized to develop models of priority.

FIG. 3 presents an example client medication management component 120 in accordance with various aspects and embodiments described herein. In the embodiment shown, the client medication management component 120 can include input reception component 302, reporting component 304, interface component 306, medication tracking component 308, prompt component 310, context component 318, and notification reception component 320. Repetitive description of like elements employed in respective embodiments is omitted for sake of brevity.

With reference to FIGS. 1, 2 and 3, in various embodiments, the client medication management component 120 can be configured to receive patient reaction information for a patient regarding the patient's reactions to a medication. As described supra with reference to FIG. 1 and system 100, this patient reaction can include information regarding a patient's reaction to a medication taken by the patient as reported to the client medication management component 120 by the patient via one or more self-reporting mechanisms, as well information regarding the patient's reaction to the medication captured by one or more medical monitoring devices 126 and/or one or more other devices 128 (e.g., physiological information, neurological information, body composition information, range of motion information, etc.). This patient reaction information can be received at the client medication management component 120 by input reception component 302.

In various embodiments, the reporting component 304 can be configured to provide the received patient reaction information to the medication management service provider 102 for processing by the server medication management component 104. For example, in response to reception of patient reaction information by the input reception component 302, the reporting component 304 can send the patient reaction information to the server medication management component 104 for incorporating into the patient's reaction log, incorporating into the historical reaction information for that medication, and/or for further processing and evaluation by the reaction evaluation component 208, the evaluation response component 214 and/or the medication optimization recommendation component 220. In some implementations, the reporting component 304 can also store the received patient reaction information in local memory of the client device 118 for usage by the additional components of the client medication management component 120.

The interface component 306 can be configured to generate a GUI that facilitates employing the various features and functionalities of the client medication management component 120 and/or the server medication management component 104. For example, in various embodiments the client medication management component 120 and/or the server medication management component 104 can provide an application (e.g., a web-application, thin client application, a mobile application, a website, etc.) that allows authorized users to access and review patient reaction logs and historical medication reaction information that is collected, collated, cleansed and indexed (e.g., by the logging component 204 and stored in one or more data stores 106). These authorized users can include for example, patients, clinicians associated with the patients, medical administrative or regulatory personnel and the like.

In some embodiments, the application can provide different levels of access and/or features and functionalities depending on the type of user. For example, in one embodiment, the application can provide different features and functionalities for patients taking medications that are being monitored by system 100 and administrative personnel (e.g., which can include for example, physicians, clinicians, regulatory agencies, etc.). According to this example embodiment, for patients, the application can allow individual patients to establish personal accounts with the medication management service provider 102. Upon performance of a suitable authentication/authorization procedure the patients can receive access to their personal medical records, including their personal reaction logs, using the application. In some implementations, the patients can also receive access to historical medication reaction information for medications they are taking and/or for one or more medications they are not taking. For patients, the application can also facilitate self-reporting of patient reaction information. For example, the interface component 306 can provide a GUI that allows the patient to provide input information identifying and/or describing the patient's reactions to a medication the patient is taking. For example, the GUI can provide for selecting a medication the patient is taking and further provide for identifying and/or describing the patient's reactions to the medication. The application can also provide patients with notifications regarding various inferences and determinations made about patient's reaction information and/or historical reaction information for a medication being taken by the patient (e.g., as determined via the reaction evaluation component 208, and the medication optimization recommendation component 220), and the like.

For administrators such as physicians, the application can allow a physician to view and review patient reaction logs of each (or in some cases one or more) of the patients the physician is treating. In some implementations, using the application, the physician can also provide feedback regarding patient reactions observed by the physician, recommendations regarding usage of a particular medication, and the like. This physician provided reaction information can also be incorporated into a patient's reaction log and/or historical reaction information for the medication. In some embodiments, the application can also allow certain physicians to provide prescriptions and/or otherwise make changes to a patient's medication regimen. The application can also provide for receiving notifications based on various inferences and determinations made about patient reaction information and/or historical reaction information for a medication (e.g., as determined via the reaction evaluation component 208, and the medication optimization recommendation component 220). For example, the application can provide physicians with notifications regarding clinically significant reactions observed for their patients, clinically signification reaction information determined for medications the physician has prescribed or may prescribe to patients, and the like.

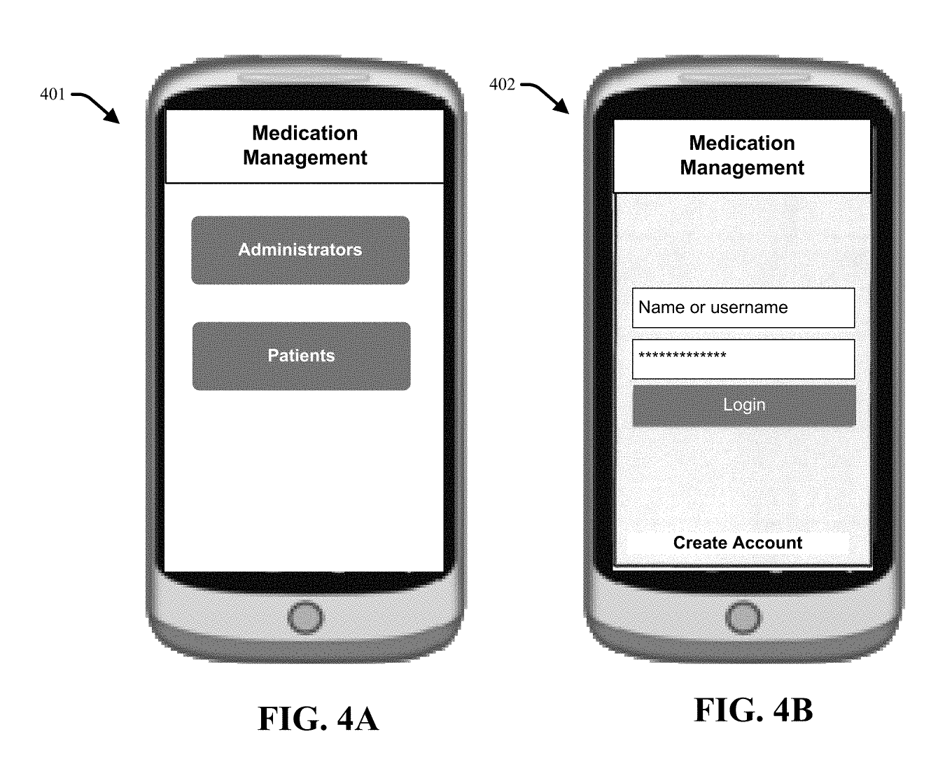

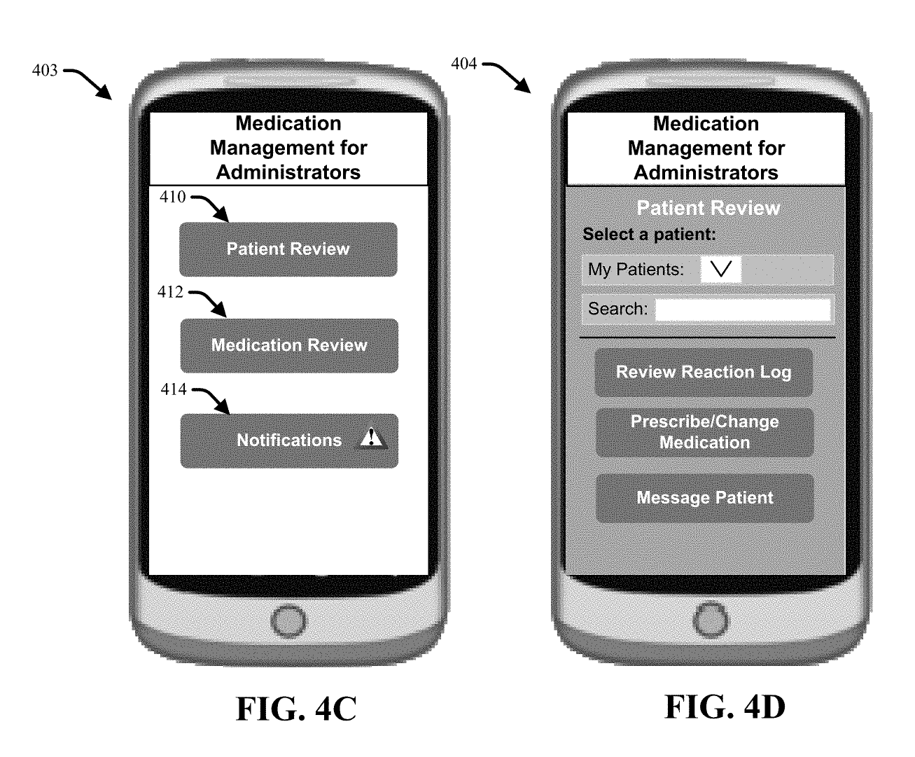

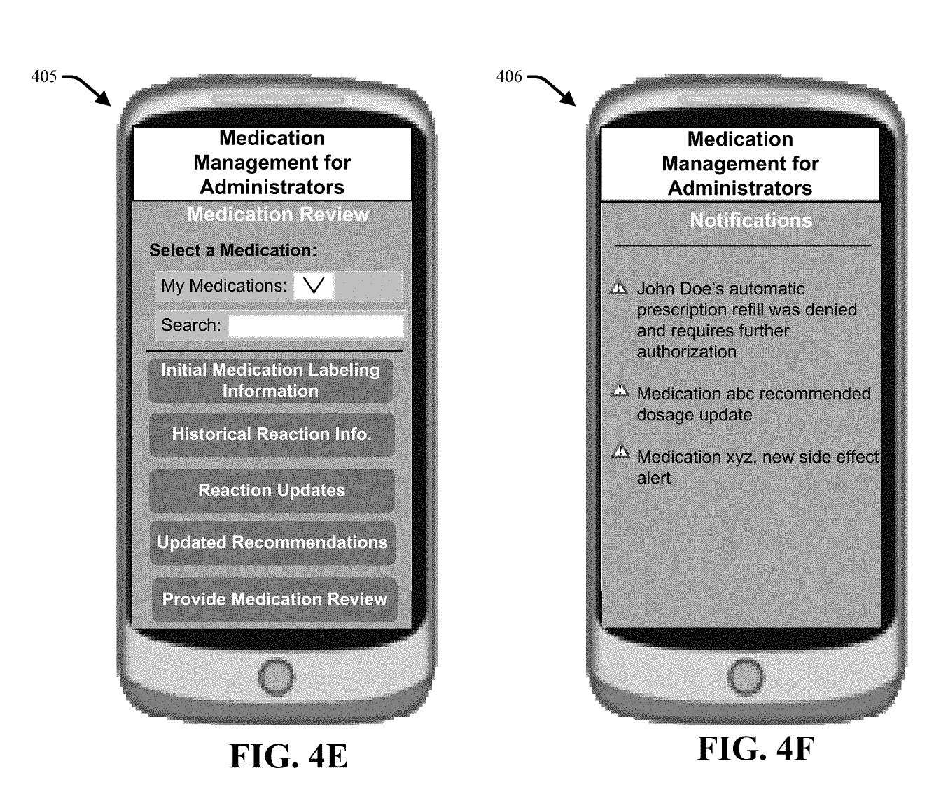

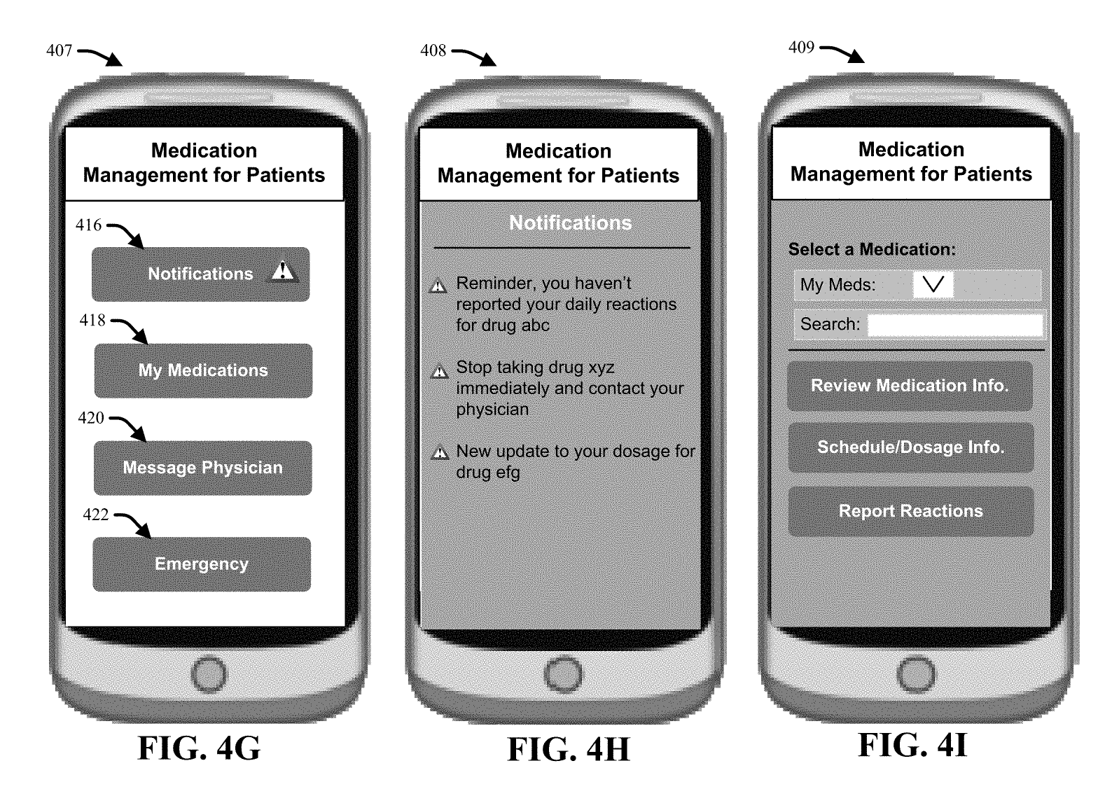

For example, FIGS. 4A-4I presents various GUIs associated with an example application provided by the client medication management component 120 and/or the server medication management component 104 that facilitates monitoring and evaluating patient reactions to medications in accordance with various aspects and embodiments described herein. In accordance with FIGS. 4A-4I, the application is referred to as a “medication management” application. In the embodiment shown, the medication management application is provided on and/or accessed via a client device (e.g., client device 118) that is a smartphone or tablet type of device. However it should be appreciated the type of client device can vary. Repetitive description of like elements employed in respective embodiments is omitted for sake of brevity.

FIG. 4A presents an initial GUI 401 that can be presented to users in association with opening of the medication management application. The initial GUI includes options to employ the features and functionalities of the medication management application as an administrator or as a patient. Selection of either of these options can provide the user with different application functionalities associated with either being an administrator or a patient. FIG. 4B presents an example login GUI 402 that can be presented to a user in response to selection of either the administrators or the patients option from GUI 401. The login GUI 402 requires the user to provide a previously established username/password combination in order to receive access to their account. If the user has not yet established an account, the login GUI provides an option for the use to select to create an account.

FIGS. 4C-4F provide example GUIs associated with usage of the medication management application as an administrator. For example, FIG. 4C depicts an example home GUI 403 for administrators in association with logging into the medication management application as an administrator. As shown in GUI 403, in one example embodiment of the medication management application for administrators, the medication management application can include a Patient Review option 410 that allows the administrator (e.g., a physician) to review information associated with the administrator's patients. The medication management application can also include a Medication Review option 412 that provides for reviewing information associated with different medications, and a Notifications option 414 that provides for reviewing notifications provided to the administrator.

FIG. 4D presents an example GUI 404 generated in response to selection of the Patient Review option 410 from GUI 403. In the embodiment shown, the patient review functionality provided by the application allows the administrator to select a patient from a pre-populated list of the administrator's patients and/or to search for patients. After a particular patient is selected, the patient review functionality provides the administrator options to review the patient's reaction log, prescribe or change the patient's medication, and send a message to the patient.

FIG. 4E presents an example GUI 405 generated in response to selection of the Medication Review option 412 from GUI 403. In the embodiment shown, the medication review functionality provided by the application allows the administrator to select a medication from a pre-populated list of medications the administrator prescribes and/or search for medications. After a particular medication is selected, the medication review functionality provides the administrator options to review the initial medication labeling information (e.g., developed based on clinical trials of the medication), review historical reaction information developed for the medication, review clinically significant updates determined for the medication (e.g., by medication evaluation component 212), review updated recommendations regarding characteristics of patients that should be prescribed the medication, dosages for the medication, and the like (e.g., as determined by the medication optimization recommendation component 220), and provide a personal review of the medication.

FIG. 4F presents an example GUI 406 generated in response to selection of the Notifications option 414 from GUI 403. Selection of the notifications option 414 presents the administrator with a list of notifications sent to the administrator. For example, in the embodiment shown, the administrator has received three notifications. The first notification informs the administrator that his patient John Doe's automatic prescription refill was denied (e.g., based on evaluation of John Doe's reaction information by the prescription component 218) and requires further authorization for fulfillment. The second notification information the administrator that medication “abc” has a recommended dosage update (e.g., as determined via the medication optimization recommendation component 220), and the third notification informs the administrator that medication “xyz” has received a new side effect alert (e.g., as determined via medication evaluation component 212).

FIGS. 4G-4I provide example GUIs associated with usage of the medication management application as patient. For example, FIG. 4G depicts an example home GUI 407 for patients in association with logging into the medication management application as a patient. As shown in GUI 407, in one example embodiment of the medication management application for patients, the medication management application can include a Notification option 416 that provides for reviewing notifications provided to the patient, and a My Medications option 418 that provides for reviewing information about the patient's medications and providing reaction information experienced by the patient in association with taking one or more medications. The medication management application can also include a Messaging option 420 to send a message to the patient's physician, and an emergency option 422 that facilitates contacting and/or initiating emergency services for the patient.

FIG. 4H presents an example GUI 408 generated in response to selection of the Notification option 416 from GUI 407. Selection of the notification option 416 presents the patient with a list of notifications sent to the patient (e.g., via notification component 216). For example, in the embodiment shown, the patient has received three notifications. The first notification reminds the patient that the patient hasn't reported his or her daily reactions for the drug “abc,” (e.g., as determined via the prompt provision component 314). The second notification information the patient to stop taking drug xyz “xyz” immediately and to contact the patient's physician (e.g., as determined via the patient evaluation component 210), and the third notification informs the patient that drug “efg” has take by the patient has an updated dosage requirement (e.g., as determined by the patient evaluation component 210 and/or the prescription component 218).

FIG. 4I presents an example GUI 409 generated in response to selection of the My Medications option 418 from GUI 407. In the embodiment shown, the My Medications functionality provided by the application allows the patient to select a medication from a pre-populated list of medications the patient is taking and/or to search for medications. After a particular medication is selected, the my medication functionality provides the patient with an option to review information about the medication (e.g., initial labeling information for the medication, clinically significant updates or notifications regarding the medication, and the like). The my medication functionality also provides the patient with an option to review the information regarding the patient's dosage and scheduling (e.g., when to take the medication) directed for the patient in association with taking the medication.

The My Medications functionalities further provides an option to report reactions experienced by the patient to the medication. For example, in one or more embodiments, selection of the report reactions option can provide the patient with a GUI that allows the patient to provide input information identifying and/or describing the patient's reactions to a medication the patient is taking. For example, the GUI can provide for identifying and/or describing the patient's reactions to the medication. In some implementations, in order to facilitate receiving patient reaction information, the GUI can include an interactive prompt or form that can be filled out by the patient (or another person that is an authorized agent of the patient). According to these embodiments, the prompt or form can identify one or more defined reactions and allow the user to provide input selecting and/or describing the particular reactions included in the prompt or form that the patient is experiencing.

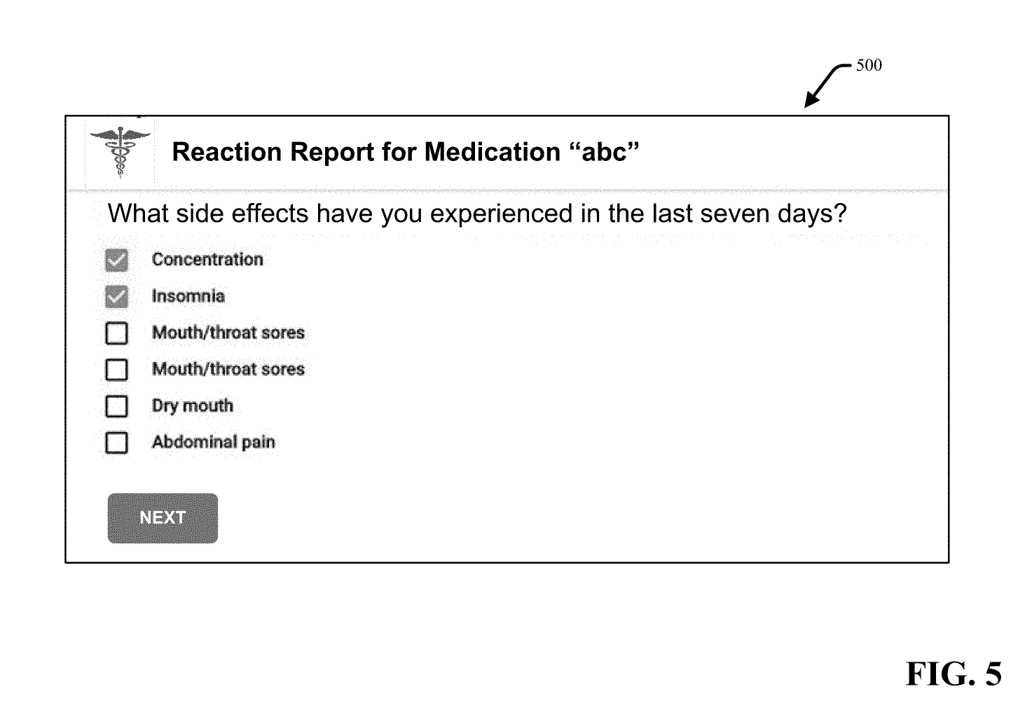

For example, FIG. 5 presents an example form 500 that facilitates reporting patient reactions to a medication in accordance with various aspects and embodiments described herein. In one or more embodiments, form 500 is a reaction report for medication “abc,” that can be presented to the patient in association with selection of the report reactions menu option from GUI 409 and selection of the drug “abc.” In the embodiment shown, the form presents the patient with a question asking the patient to check the boxes corresponding to the side effects the patient has experienced in the last seven days. The bottom of the form has a next button. In an aspect, selection of the next button can result in the presentation of one or more additional questions to the patient regarding the reactions experienced by the patient. It should be appreciated that form 500 is merely exemplary and that the selection of reactions via check boxes is one mechanism for facilitating collection of patient feedback regarding reactions experienced by the patient. For example, in other embodiments, other type of visual input and selection icons can be employed to facilitate receiving reaction input (e.g., selection of images, marking points or locations on a visual representation or avatar corresponding to the patient, using a sliding scale to provide input regarding intensity, voice input, gesture input, etc.).

With reference back to FIG. 3, in one or more embodiments, the prompt component 310 can facilitate self-reporting of patient reaction information associated with a particular medication. For example, in some embodiments, the prompt component 310 can generate an interactive prompt or form (e.g., form 500) that can be rendered via the GUI. The interactive prompt or form can comprise one or more reactions which the patient can select to indicate the particular reactions the patient is experiencing or has experienced in association with taking a particular medication. In some implementations, depending on the type of reaction, the prompt or form can also provide for describing one or more additional qualitative and/or quantitative characteristics associated with the reaction. For example, the one or more additional characteristics can relate to a severity or intensity of the reaction, a frequency of the reaction, duration of the reaction, a physical location of the reaction relative to the body, an appearance of the reaction (e.g. if the reaction is visible on the body of the patient), and the like.

In other embodiments, rather than having the patient fill out an interactive prompt or form via interaction with the GUI, the prompt component 310 can provide the patient with spoken questions regarding the patient's reactions to a medication that the patient can also respond to with speech answers. For example, the prompt component 310 can ask the user audible questions regarding possible reactions the patient is having and the user can answer the questions with speech that can be received by the input component 122 and converted to text (e.g. using speech-to-text processing hardware/software). In other embodiments, the client medication management component 120 can also provide for reception of a free form description of the patient's reactions (e.g. provided via text input, speech-to-text input, or the like). For example, in some implementations, a patient may experience a reaction that is not included in a prompt or form and/or is otherwise not presented to the patient. According to these implementations, the patient can provide additional information describing the patient's reactions.

In the embodiment shown, the prompt component 310 can include prompt generation component 312 to provide for generating a prompt (e.g., a visual or audible prompt) or form that facilitates reporting patient reaction information. In this regard, the prompt generation component 312 can control the content (e.g., the questions) that are included in a prompt or form. For example, the prompt generation component 312 can determine what questions to include in a prompt or form regarding patient reactions to a particular medication. For instance, the prompt generation component 312 can determine one or more defined reactions to ask a patient if they are experiencing. In some implementations, depending on the type of reaction and/or the answer provided by the patient affirming experience of a particular reaction, the prompt generation component 312 can also determine one or more additional qualitative and/or quantitative characteristics associated with the reaction to ask the patient. For example, the one or more additional characteristics can relate to a severity or intensity of the reaction, a frequency of the reaction, duration of the reaction, a physical location of the reaction relative to the body, an appearance of the reaction (e.g. if the reaction is visible on the body of the patient), and the like. For example, in response to reception of user input affirming the patient has experienced pain, the prompt generation component 312 can be configured to further ask the patient to rate the pain on a scale of 1 to 10, wherein 10 represents the highest level of pain and 1 represents the lowest level of pain.

In various implementations, the prompt generation component 312 can determine the questions for inclusion in a prompt or form provided to a patient based on the particular medication the patient is taking. In this regard, the questions can vary depending on the medication. For example, the one or more defined reactions can include side effects and/or intended effects of the medication determined in association with clinical trials for the medication. According to this example, the prompt generation component 312 can access or employ a previously generated prompt associated with the medication including the specific questions to ask the patient via the prompt or form. For example, each (e.g., or one more) medication monitored by system 100 can be associated with previously generated prompt or form including a defined set of reaction questions for the medication. Alternatively, the medication can be associated with a defined set of reaction questions and/or toxicities (e.g., side effects) that can be linked from a database (e.g., one or more data stores 106 including medication information 110 that provides initial labeling information for the medication). According to this implementation, the prompt generation component 312 can access the database to select and incorporate the appropriate toxicity questions for the medication in a prompt or form that is presented to the patient.

In another example, the prompt generation component 312 can include questions in a prompt or form regarding patient reactions based on one or more reactions and/or questions about the reactions selected by a clinician or physician with respect to a particular medication. According to this example, the clinician or physician can provide input (e.g., using the application provided by the client medication management component 120) selecting one or more reactions and/or questions about the reactions the clinician or physician is interested in evaluating in association with a particular drug. The prompt generation component 312 can further include the clinician selected reactions in the prompt or form that it provides to the patient. In another implementation, the prompt generation component 312 can generate a prompt comprising a laundry list of many possible reactions (e.g., side effects). For example, the laundry list can include reactions observed by patients taking the medication or similar medications in the past, (e.g., including common and uncommon reactions). In another example, the laundry list can include a variety of possible reactions that have been observed for a variety of different medications over time. According to this implementation, the patient can select only those reactions that apply to the patient that the patient is or has experienced.

In one implementation, these reactions evaluated by a prompt or form generated by the prompt generation component 312 can include additional reactions not observed in clinical trials for the medication but observed based on feedback received by the server medication management component 104 from patients monitored after clinical trials. For example, based on detection by the medication evaluation component 212 of a new (e.g., not observed in clinical trials and/or included in the medication labeling information) patient reaction to a medication or detection (e.g., based on a threshold number of patients experiencing this new reaction), the evaluation response component 214 can be configured to instruct the prompt generation component 312 to include one or more questions regarding this new reaction in a prompt presented to patients taking the medication.

The prompt component 310 can also include prompt provision component 314 to facilitate timing of provision of the prompt or form to the patient (e.g., whether it is a visual interactive prompt such as a form, audible questions, or the like). For example, in some embodiments, the patient reaction information can be collected over a period of time to facilitate tracking and monitoring of the patient's reactions to a medication taken by the patient over the period of time. With these embodiments, the patient reaction information can be collected on a regular basis over the period of time the patient takes the medication and/or or for a defined period of time after the patient stops taking the medication. In one implementation, the patient reaction information can be collected over this period of time according to a defined schedule (e.g. once an hour, once a day, once a month). According to this implementation, the prompt provision component 314 can be configured to provide the patient with the prompt or form in accordance with the defined schedule. In another embodiment, the patient can be instructed by the prompt provision component 314 to provide reaction information at will (e.g., using a prompt or form and/or in free form) in accordance with a defined schedule (e.g., once a day, once a week, once a month, etc.)

In some implementations, if the patient does not provide the patient reaction information in accordance with the defined schedule, the prompt provision component 314 can further generate and provide the patient with a notification reminding the patient to report their reaction information. For example, in some implementations, the notification can include a pop-up or other type of notification with a link to open and respond to a prompt of form including questions for the patient to respond to regarding the patient's reaction to a particular medication. In other implementations, the patient reaction information can be collected over this period of time at points when the patient notices or experiences the reaction. For example, the patient can be directed to report the patient's side effects on demand or at will in response to recognition of possible side effects.

The prompt component 310 can also include prompt tailoring component 316 to provide for tailoring of the content (e.g., the questions) included in the prompt or form provided to the patient and/or the timing of the provision of the prompt or form based on the various parameters associated with the patient and a current context of the patient. For example, in some embodiments, the prompt tailoring component 316 can tailor the particular reactions and/or questions about the reactions included in a prompt or form provided to the patient based on one or more personal characteristics of the patient, including but not limited to: one or more demographic characteristics of the patient (e.g., gender, age, ethnicity, etc.), one or more characteristics pertaining to the medical health history of the patient (e.g., previous medical conditions, hereditary medical conditions, patient blood type, etc.), one or more characteristics regarding the current health state of the patient (e.g., one or more medical conditions currently affecting the patient, one or more other medications the patient is taking, whether the patient is pregnant, patient weight, etc.,) and the like. For example, certain side effects may only be attributed to women. In this regard, the prompt tailoring component 316 can exclude questions about these women specific side effects from prompts or forms presented to men. With these embodiments, the prompt tailoring component 316 can access or receive information regarding these types of personal characteristics associated with the patient at the medication management service provider 102 (e.g., in the patient information 108 provided at the one or more data stores 106) and/or at one or more external systems/sources 116.

In some embodiments, the prompt tailoring component 316 can also tailor the content included in a prompt or form presented to the patient based on received physiological information for the patient from one or more medical monitoring devices 126. In this regard, based on reception of physiological information for a patient that has been determined to indicate one or more defined reactions to a medication the patient is taking, prompt tailoring component 316 can be configured to include one or more questions about the one or more defined reactions in a prompt or form that is presented to the patient. For example, based on reception of physiological information for a patient indicating the patient's body temperature is above a threshold level, the prompt tailoring component 316 can be configured to incorporate questions in a prompt or form presented to a patient regarding other types of patient reactions associated with high body temperature (e.g., lightheadedness, dizziness, aches and pains, etc.).

The prompt tailoring component 316 can also tailor the content (e.g., the questions) included in a prompt or form presented to a patient based on previously received reaction information for the patient. For example, in some implementations, if the patient previously indicated the patient has not experienced certain reactions, the prompt tailoring component 316 can exclude questions regarding these reactions in future prompts/forms presented to the patient. In another example, if the patient previously indicated that the patient had experienced a particular reaction in the past, the prompt tailoring component 316 can include follow up questions regarding the particular reaction in subsequent prompts/forms presented to the patient. In another implementation, based on one or more previously experienced reactions reported by the patient, the prompt tailoring component 316 can identify one or more additional reactions or additional characteristics about the previous reactions that have been correlated to the previous reactions. According to this implementation, the prompt tailoring component 316 can incorporate one or more additional questions in a subsequent prompt or form that is presented to the patient regarding the one or more additional reactions or additional characteristics about the previous reactions that have been correlated to the previous reactions. With these implementations, the prompt tailoring component 316 can access and employ predefined information (e.g., provided in memory at the client device 118, provided by the medication management service provider 102, and/or provided at one or more external systems/sources 116) identifying additional questions regarding reactions that have been correlated to other reactions. For example, based on received input indicating the patient is experiencing pain associated with a particular area of the body after taking a particular medication, the prompt tailoring component 316 can access information that identifies one or more additional reactions and/or characteristics associated with pain in the particular area of the body (e.g., inflammation). The prompt tailoring component 316 can further incorporate the one or more questions in a subsequent prompt or form presented to the patient regarding the one or more additional reactions and/or characteristics (e.g., a question asking the patient if he or she is also experiencing inflammation in the area).

In addition to tailoring prompt content based on personal aspects associated with the patient and/or previously provided reaction information provided by the patient, the prompt tailoring component 316 can also be configured to tailor the timing of provision of prompts or forms to the patient based a context of the patient. For example, the patient may be more inclined to respond and/or accurately respond to a prompt or form regarding the patient's reactions to a particular medication at in certain contexts, such as at certain times of day (e.g., in the morning), at certain physical locations (e.g., at home), when the patient is not engaged in a distracting activity, when the patient is sedentary, and the like. Accordingly, in some embodiments, the client medication management component 120 can include context component 318 to facilitate determining various contextual factors associated with the patient and/or the patient's usage of the client device to determine optimal points in time to present the patient with a prompt or form asking the patient to report his or her reaction information. For example, in one or more embodiments, the context component can determine regarding time of day, location of the patient, and mobility state of the patient (e.g., walking, running, driving, sitting, standing still, etc.). The context component can employ various known techniques to determine the location of the patient (e.g., GPS) and the mobility state of the patient (e.g., motion sensors, camera based motion detection). In some implementations, the context component 318 can also determine contextual information regarding an activity being performed by the patient (e.g., including physical activities and/or activities associated with usage of the client device such as browsing the web, watching a video, conducting a phone call, etc.). In some implementations, the context component 318 can access information regarding a patients schedule to facilitate determining activities performed by the patient. The prompt tailoring component 316 can further tailor the timing of provision of a prompt or form to the patient in contexts previously determined to be contexts in which the patient is likely to respond and/or accurately respond to the prompt or form.

In some implementations, the prompt tailoring component 316 can also tailor timing of provision of a prompt or form to a patient based when the patient is experiencing certain physiological symptoms (as detected via one or more medical monitoring devices 126). According to these implementations, in response to reception of physiological data from one or more medical monitoring devices 126 (or psychological data, body composition data and the like from one or more other devices 128), the prompt tailoring component 316 can be configured to direct the prompt provision component 314 to provide the patient with a prompt or form regarding the patient's reactions to a medication. For example, based on reception of physiological data indicating the patient's blood pressure is low, the prompt tailoring component 316 can direct the prompt provision component 314 to provide the patient with a prompt asking the patient the following: “Are you feeling OK?, you blood pressure is quite low.” In response to patient input indicating the patient is not feeling “OK,” the prompt provision component 314 can subsequently provide the patient with additional questions regarding some possible reactions to the medication taken by the patient that the patient may be experiencing.

Still in one or more additional embodiments, the prompt tailoring component 316 can be configured to tailor provision of a prompt or form to a patient regarding the patient's reaction to a medication based on when the patient takes his or her medication. For example, the prompt tailoring component 316 can be configured to present the patient with a prompt or form asking the patient if the patient is experiencing certain reactions to the medication at one or more defined points in time after the patient takes the medication (e.g., after ten minutes of taking the medication, after an hour of taking the medication, after twenty four hours of taking the medication, after a week of taking the medication, etc.). The one or more points in time at which the patient is asked about his or her reactions to the medication can vary based on the particular medication.

With these embodiments, in some implementation, the prompt tailoring component 316 can determine when the patient takes his or her medication according to a defined medication regimen followed by the patient (e.g., as provided with the patient information 108 included in the one or more data stores). In this regard, based on information stating that the patient has been instructed to take a particular medication each day at 10 am and 8 pm, the prompt tailoring component 316 can assume the patient takes his or her medication each day at 10 am and 8 pm. In some implementations, the client medication management component 120 can include a medication tracking component 308 to facilitate ensuring a patient takes his or her medication as prescribed. In this regard, the medication tracking component 308 can employ and/or provide one or more tools to ensure the patient takes his or her medication as directed and to receive information from the patient confirming the patient has in fact taken his or her medication. Accordingly, the medication tracking component 308 can provide the prompt tailoring component 316 with information confirming when the patient has taken a particular medication. The prompt tailoring component 316 can then accurately determine when to provide the patient with a prompt or form regarding the patient's reaction to the medication based on when the patient has actually taken the medication.

The client medication management component 120 can also include notification reception component 320. The notification reception component 320 can be configured to receive and present notifications to users of the client medication management component 120 (or application provided thereby). For example, the notification reception component 320 can be configured to provide patients with notification received from the server medication management component 104 in response to determinations and/or inferences made by the reaction evaluation component 208. For example, the notification reception component 320 can be configured to receive and present a notification from the server medication management component 104 in response to a determination that the patient has experienced or is experiencing a clinically significant reaction (e.g., a harmful side effect). In some implementations, the notification can include information instructing the patient regarding an action to perform to stop or minimize the side effect. In another implementation, the notification can include information instructing the patient regarding a change to the patient's medication regimen determined based on the side effect.

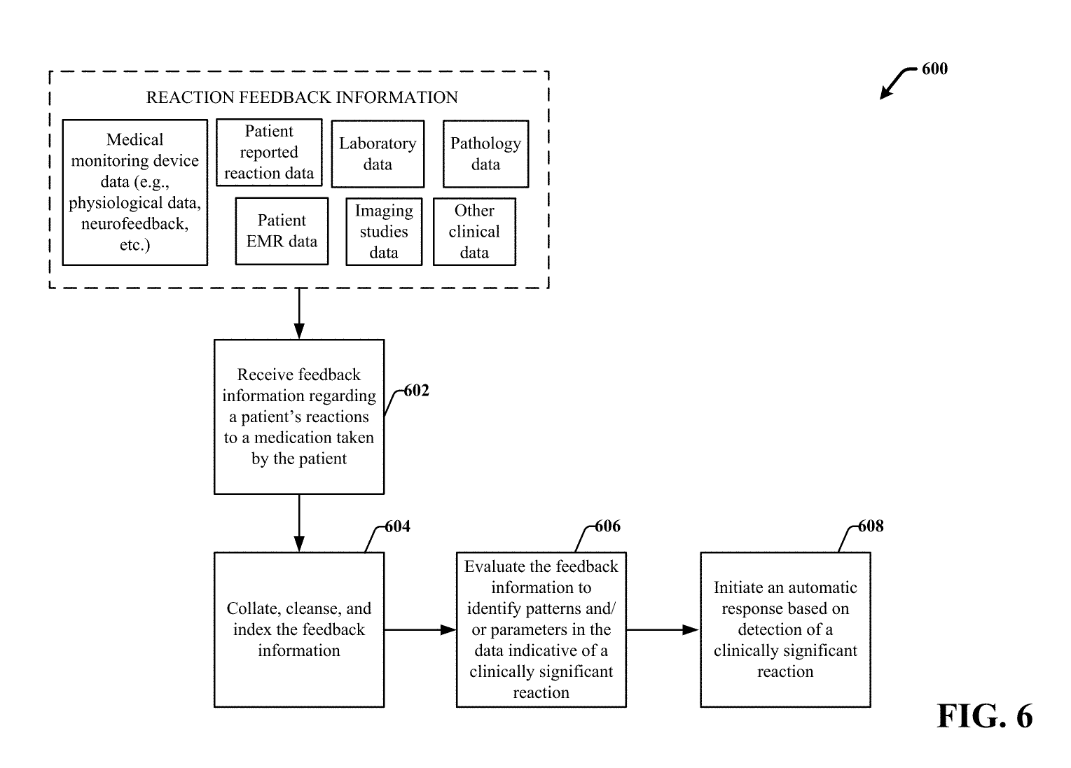

FIGS. 6-9 illustrate various methodologies in accordance with the disclosed subject matter. While, for purposes of simplicity of explanation, the methodologies are shown and described as a series of acts, it is to be understood and appreciated that the disclosed subject matter is not limited by the order of acts, as some acts can occur in different orders and/or concurrently with other acts from that shown and described herein. For example, those skilled in the art will understand and appreciate that a methodology could alternatively be represented as a series of interrelated states or events, such as in a state diagram. Moreover, not all illustrated acts can be required to implement a methodology in accordance with the disclosed subject matter. Additionally, it should be further appreciated that the methodologies disclosed hereinafter and throughout this specification are capable of being stored on an article of manufacture to facilitate transporting and transferring such methodologies to computers.

FIG. 6 provides a high level flow diagram of an example computer-implemented method 600 that facilitates monitoring and evaluating patient reactions to medications in accordance with various aspects and embodiments described herein. In accordance with various embodiments, method 600 demonstrates a high level process that can be performed by the server medication management component 104 in connection with monitoring and evaluating patient reactions to medications. Repetitive description of like elements employed in respective embodiments is omitted for sake of brevity.

At 602, feedback information regarding a patient's reactions to a medication take by the patient is received (e.g., via reaction information reception component 202). This feedback information can be received regularly or continuously over a period of time the patient is taking the medication (and/or after the patient stops taking the medication) from various sources. For example, in the embodiment shown, the feedback information can include data received from one or more medical monitoring devices, patient reported reaction data, laboratory data, pathology data, data included in the patient's EMR, imaging studies data, and other suitable clinical data related to the patient's reactions to the medication. At 604, the feedback information can be collated, cleansed and indexed to facilitate evaluation of the feedback information (e.g., via logging component 204).

At 606, the feedback information can be evaluated (e.g., using one or more defined algorithms and/or mathematical models) to identify parameters and/or parameters in the data indicative of a clinically significant reaction (e.g., via reaction evaluation component 208). For example, the feedback information can be compared to reference information (e.g., included with the response information 112) that identifies defined parameters, values, patterns, etc., that are considered indicative of a clinically significant reaction. For instance, reaction information that indicates a patient has had a particular reaction (e.g., a fever) for a threshold period of time (e.g., 24 hours) can be considered a clinically significant reaction. At 608, an automatic response can be initiated based on detection of a clinically significant reaction (e.g., via evaluation response component 214). For example, in some implementations, the automatic response can include generating and sending a notification (e.g., via notification component 216) to an appropriate entity (e.g., the patient's physician) regarding the patient's reaction. In another implementation, the automatic response can include adapting the patient's medication regimen (e.g., via prescription component 218) and notifying the patient (e.g., via notification component 216) regarding the change to the patient's medication regimen. The patients EMR can further be updated to reflect the change to the patient's medication regimen. In some implementations, the reason for the change (e.g., the clinically signification reaction detected) can also be recorded in the patient's EMR.

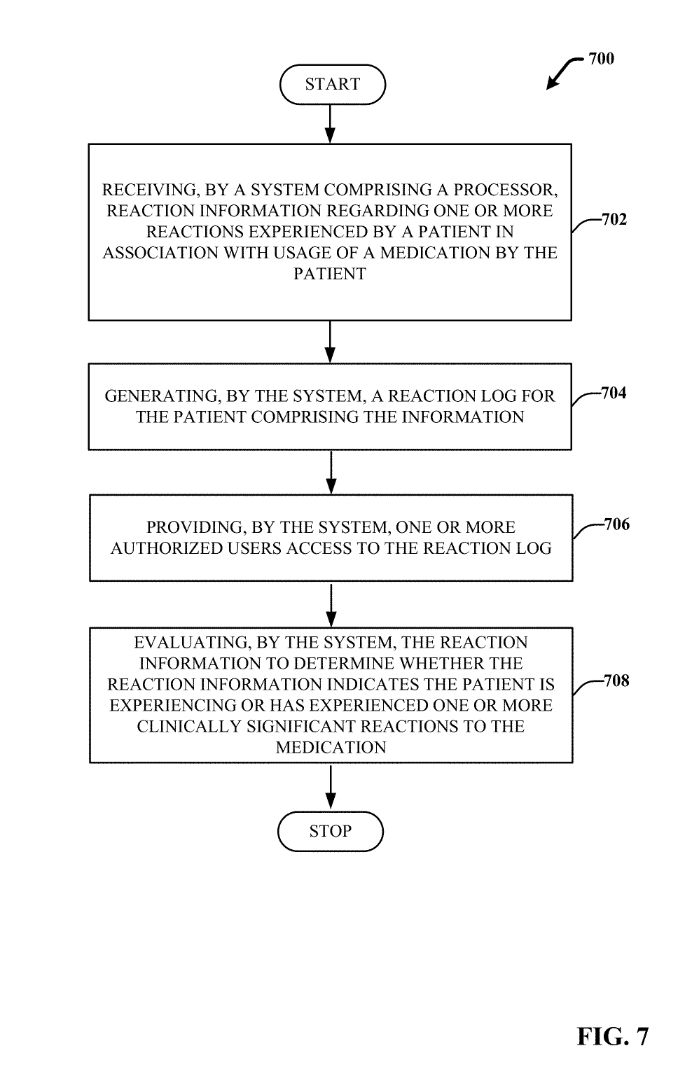

FIG. 7 provides a flow diagram of an example computer-implemented method 700 that facilitates monitoring and evaluating patient reactions to medications in accordance with various aspects and embodiments described herein. Repetitive description of like elements employed in respective embodiments is omitted for sake of brevity.

At 702 a system comprising a processor (e.g., system 100), receives reaction information regarding one or more reactions experienced by a patient in association with usage of a medication by the patient (e.g., via reaction information reception component 202). At 704, the system generates a reaction log for the patient comprising the information (e.g., via logging component 204 and/or report component 206). At 706, the system provides one or more authorized users access to the reaction log, and at 708, the system evaluates the reaction information to determine whether the reaction information indicates the patient is experiencing or has experienced one or more clinically significant reactions to the medication.

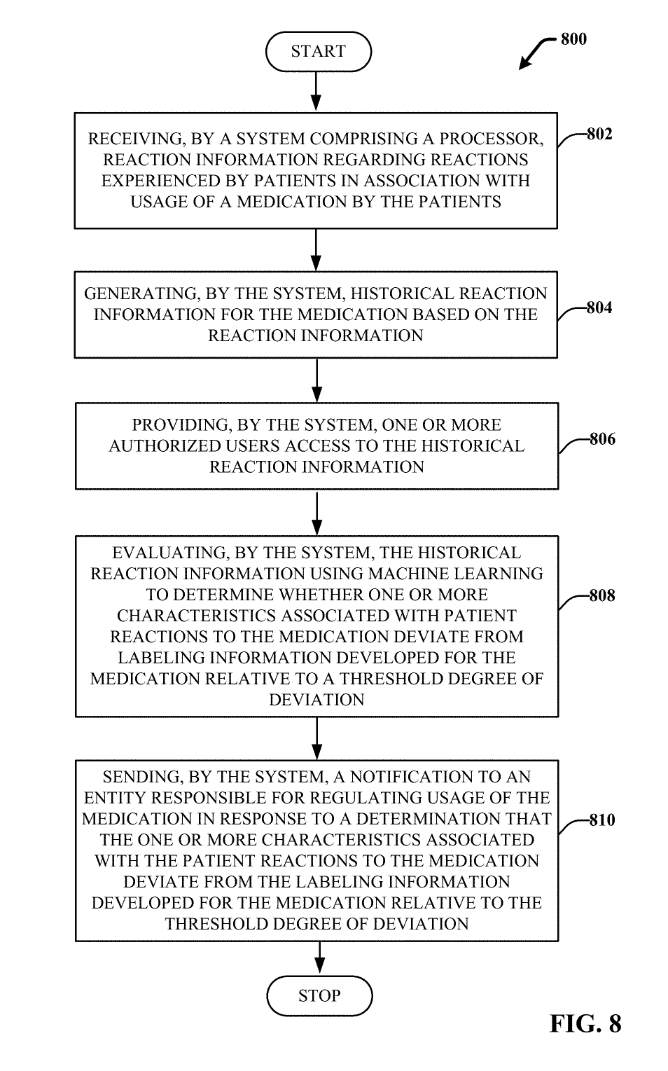

FIG. 8 provides a flow diagram of another example computer-implemented method 800 that facilitates monitoring and evaluating patient reactions to medications in accordance with various aspects and embodiments described herein. Repetitive description of like elements employed in respective embodiments is omitted for sake of brevity.

At 802 a system comprising a processor (e.g., system 100), receives reaction information regarding reactions experienced by patients in association with usage of a medication by the patients (e.g., via reaction information reception component 202). At 804, the system generates a historical reaction information for the medication based on the reaction information comprising the information (e.g., via logging component 204 and/or report component 206). At 806, the system provides one or more authorized users access to the historical reaction information. At 808, the system evaluates the historical reaction information using machine learning to determine whether one or more characteristics associated with patient reactions to the medication deviate from labeling information developed for the medication relative to a threshold degree of deviation (e.g., via medication evaluation component 212). At 810, the system sends a notification to an entity responsible for regulating usage of the medication (e.g., the FDA) in response to a determination that the one or more characteristics associated with the patient reactions to the medication deviate from the labeling information developed for the medication relative to the threshold degree of deviation (e.g., via notification component 216).

FIG. 9 provides a flow diagram of another example computer-implemented method 900 that facilitates monitoring and evaluating patient reactions to medications in accordance with various aspects and embodiments described herein. Repetitive description of like elements employed in respective embodiments is omitted for sake of brevity.

At 902, a client device (e.g., client device 118) operatively coupled to a processor, generates a prompt requesting information regarding one or more reactions experienced by a patient in association with usage of a medication by the patient (e.g., via prompt component 310). At 904, the client device receives first information regarding the one or more reactions experienced by the patient in response to the generating the prompt (e.g., via input reception component 302). At 906, the client device reports the first information to a server device (e.g., the medication management service provider 102) in response to reception of the first information (e.g., via reporting component 304), wherein based on provision of the first information to the server device, the server device is configured to store the first information in a reaction log for the patient and provide one or more authorized users access to the reaction log.

### Example Operating Environments

The systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which may be explicitly illustrated in this disclosure.

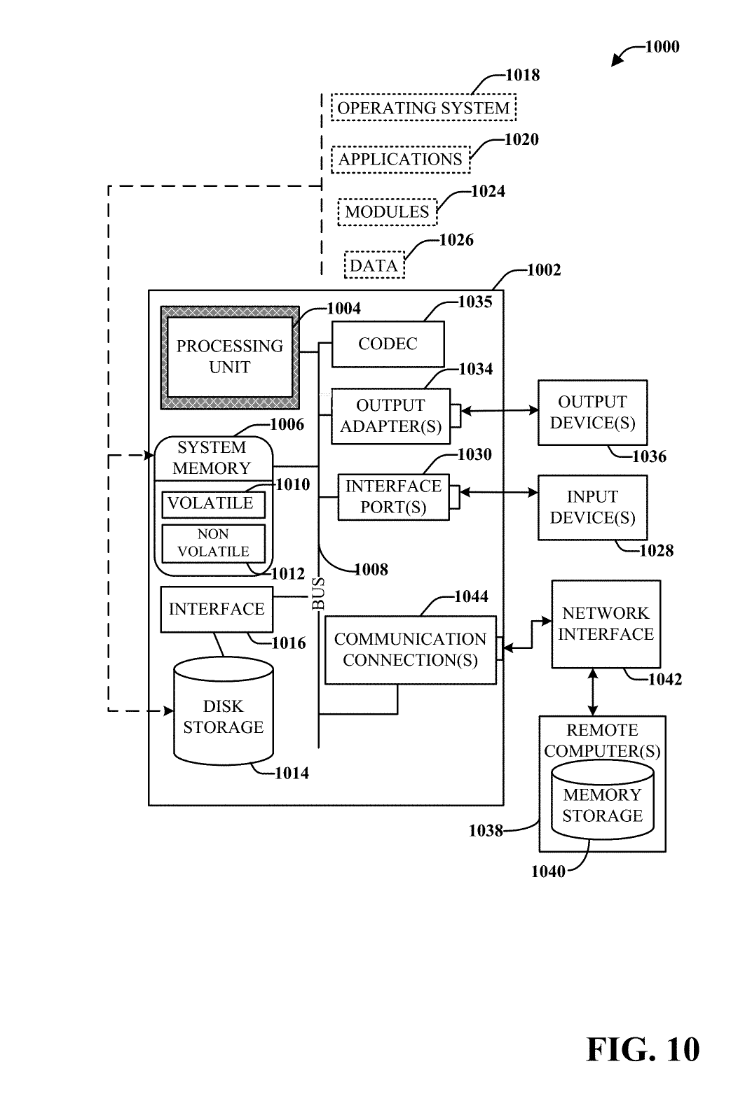

With reference to FIG. 10, a suitable environment 1000 for implementing various aspects of the claimed subject matter includes a computer 1002. The computer 1002 includes a processing unit 1004, a system memory 1006, a codec 1005, and a system bus 1008. The system bus 1008 couples system components including, but not limited to, the system memory 1006 to the processing unit 1004. The processing unit 1004 can be any of various available suitable processors. Dual microprocessors and other multiprocessor architectures also can be employed as the processing unit 1004.

The system bus 1008 can be any of several types of suitable bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 10104), and Small Computer Systems Interface (SCSI).

The system memory 1006 includes volatile memory 1010 and non-volatile memory 1012. The basic input/output system (BIOS), containing the basic routines to transfer information between elements within the computer 1002, such as during start-up, is stored in non-volatile memory 1012. In addition, according to present innovations, codec 1005 may include at least one of an encoder or decoder, wherein the at least one of an encoder or decoder may consist of hardware, a combination of hardware and software, or software. Although, codec 1005 is depicted as a separate component, codec 1005 may be contained within non-volatile memory 1012. By way of illustration, and not limitation, non-volatile memory 1012 can include read only memory (ROM), programmable ROM (PROM), electrically programmable ROM (EPROM), electrically erasable programmable ROM (EEPROM), or flash memory. Volatile memory 1010 includes random access memory (RAM), which acts as external cache memory. According to present aspects, the volatile memory may store the write operation retry logic (not shown in FIG. 10) and the like. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM.

Computer 1002 may also include removable/non-removable, volatile/non-volatile computer storage medium. FIG. 10 illustrates, for example, disk storage 1014. Disk storage 1014 includes, but is not limited to, devices like a magnetic disk drive, solid state disk (SSD) floppy disk drive, tape drive, Jaz drive, Zip drive, LS-70 drive, flash memory card, or memory stick. In addition, disk storage 1014 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 1014 to the system bus 1008, a removable or non-removable interface is typically used, such as interface 1016.

It is to be appreciated that FIG. 10 describes software that acts as an intermediary between users and the basic computer resources described in the suitable operating environment 1000. Such software includes an operating system 1018. Operating system 1018, which can be stored on disk storage 1014, acts to control and allocate resources of the computer system 1002. Applications 1020 take advantage of the management of resources by operating system 1018 through program modules 1024, and program data 1026, such as the boot/shutdown transaction table and the like, stored either in system memory 1006 or on disk storage 1014. It is to be appreciated that the claimed subject matter can be implemented with various operating systems or combinations of operating systems.

A user enters commands or information into the computer 1002 through input device(s) 1028. Input devices 1028 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1004 through the system bus 1008 via interface port(s) 1030. Interface port(s) 1030 include, for example, a serial port, a parallel port, a game port, and a universal serial bus (USB). Output device(s) 1036 use some of the same type of ports as input device(s). Thus, for example, a USB port may be used to provide input to computer 1002, and to output information from computer 1002 to an output device 1036. Output adapter 1034 is provided to illustrate that there are some output devices 1036 like monitors, speakers, and printers, among other output devices 1036, which require special adapters. The output adapters 1034 include, by way of illustration and not limitation, video and sound cards that provide a means of connection between the output device 1036 and the system bus 1008. It should be noted that other devices and/or systems of devices provide both input and output capabilities such as remote computer(s) 1038.

Computer 1002 can operate in a networked environment using logical connections to one or more remote computers, such as remote computer(s) 1038. The remote computer(s) 1038 can be a personal computer, a server, a router, a network PC, a workstation, a microprocessor based appliance, a peer device, a smart phone, a tablet, or other network node, and typically includes many of the elements described relative to computer 1002. For purposes of brevity, only a memory storage device 1040 is illustrated with remote computer(s) 1038. Remote computer(s) 1038 is logically connected to computer 1002 through a network interface 1042 and then connected via communication connection(s) 1044. Network interface 1042 encompasses wire and/or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 1044 refers to the hardware/software employed to connect the network interface 1042 to the bus 1008. While communication connection 1044 is shown for illustrative clarity inside computer 1002, it can also be external to computer 1002. The hardware/software necessary for connection to the network interface 1042 includes, for exemplary purposes only, internal and external technologies such as, modems including regular telephone grade modems, cable modems and DSL modems, ISDN adapters, and wired and wireless Ethernet cards, hubs, and routers.

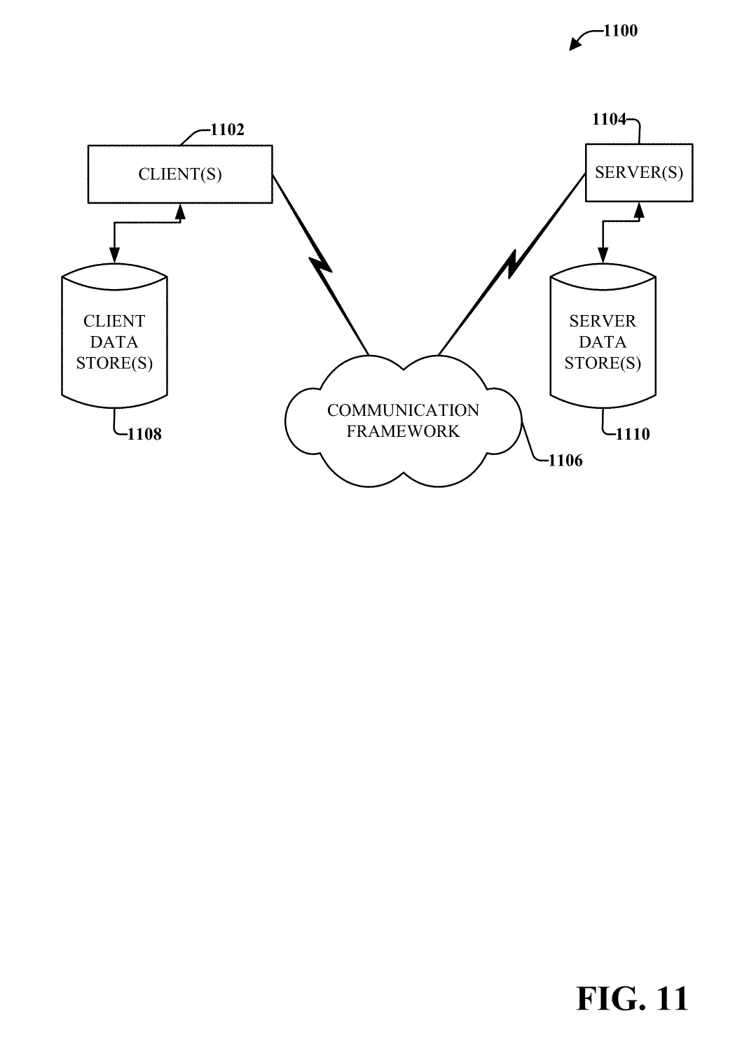

Referring now to FIG. 11, there is illustrated a schematic block diagram of a computing environment 1100 in accordance with this disclosure. The environment 1110 includes one or more client(s) 1102 (e.g. laptops, smart phones, PDAs, media players, computers, portable electronic devices, tablets, and the like). The client(s) 1102 can be hardware and/or software (e.g. threads, processes, computing devices). The system 1100 also includes one or more server(s) 1104. The server(s) 1104 can also be hardware or hardware in combination with software (e.g. threads, processes, computing devices). The servers 1104 can house threads to perform transformations by employing aspects of this disclosure, for example. One possible communication between a client 1102 and a server 1104 can be in the form of a data packet transmitted between two or more computer processes wherein the data packet may include video data. The data packet can include a metadata, e.g. associated contextual information, for example. The system 1100 includes a communication framework 1106 (e.g. a global communication network such as the Internet, or mobile network(s)) that can be employed to facilitate communications between the client(s) 1102 and the server(s) 1104.

Communications can be facilitated via a wired (including optical fiber) and/or wireless technology. The client(s) 1102 include or are operatively connected to one or more client data store(s) 1108 that can be employed to store information local to the client(s) 1102 (e.g. associated contextual information). Similarly, the server(s) 1104 are operatively include or are operatively connected to one or more server data store(s) 1110 that can be employed to store information local to the servers 1104.

In one embodiment, a client 1102 can transfer an encoded file, in accordance with the disclosed subject matter, to server 1104. Server 1104 can store the file, decode the file, or transmit the file to another client 1102. It is to be appreciated, that a client 1102 can also transfer uncompressed file to a server 1104 and server 1104 can compress the file in accordance with the disclosed subject matter. Likewise, server 1104 can encode video information and transmit the information via communication framework 1100 to one or more clients 1102.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described in this description can include electrical circuit(s) that can include components and circuitry elements of suitable value in order to implement the embodiments of the subject innovation(s). Furthermore, it can be appreciated that many of the various components can be implemented on one or more integrated circuit (IC) chips. For example, in one embodiment, a set of components can be implemented in a single IC chip. In other embodiments, one or more of respective components are fabricated or implemented on separate IC chips.

What has been described above includes examples of the embodiments of the present invention. It is, of course, not possible to describe every conceivable combination of components or methodologies for purposes of describing the claimed subject matter, but it is to be appreciated that many further combinations and permutations of the subject innovation are possible. Accordingly, the claimed subject matter is intended to embrace all such alterations, modifications, and variations that fall within the spirit and scope of the appended claims. Moreover, the above description of illustrated embodiments of the subject disclosure, including what is described in the Abstract, is not intended to be exhaustive or to limit the disclosed embodiments to the precise forms disclosed. While specific embodiments and examples are described in this disclosure for illustrative purposes, various modifications are possible that are considered within the scope of such embodiments and examples, as those skilled in the relevant art can recognize.

In particular and in regard to the various functions performed by the above described components, devices, circuits, systems and the like, the terms used to describe such components are intended to correspond, unless otherwise indicated, to any component which performs the specified function of the described component (e.g. a functional equivalent), even though not structurally equivalent to the disclosed structure, which performs the function in the disclosure illustrated exemplary aspects of the claimed subject matter. In this regard, it will also be recognized that the innovation includes a system as well as a computer-readable storage medium having computer-executable instructions for performing the acts and/or events of the various methods of the claimed subject matter.

The aforementioned systems/circuits/modules have been described with respect to interaction between several components/blocks. It can be appreciated that such systems/circuits and components/blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described in this disclosure may also interact with one or more other components not specifically described in this disclosure but known by those of skill in the art.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

As used in this application, the terms “component,” “module,” “system,” or the like are generally intended to refer to a computer-related entity, either hardware (e.g. a circuit), a combination of hardware and software, software, or an entity related to an operational machine with one or more specific functionalities. For example, a component may be, but is not limited to being, a process running on a processor (e.g. digital signal processor), a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer readable storage medium; software transmitted on a computer readable transmission medium; or a combination thereof.

Moreover, the words “example” or “exemplary” are used in this disclosure to mean serving as an example, instance, or illustration. Any aspect or design described in this disclosure as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Computing devices typically include a variety of media, which can include computer-readable storage media and/or communications media, in which these two terms are used in this description differently from one another as follows. Computer-readable storage media can be any available storage media that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by one or more local or remote computing devices, e.g. via access requests, queries or other data retrieval protocols, for a variety of operations with respect to the information stored by the medium.

On the other hand, communications media typically embody computer-readable instructions, data structures, program modules or other structured or unstructured data in a data signal that can be transitory such as a modulated data signal, e.g. a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

In view of the exemplary systems described above, methodologies that may be implemented in accordance with the described subject matter will be better appreciated with reference to the flowcharts of the various figures. For simplicity of explanation, the methodologies are depicted and described as a series of acts. However, acts in accordance with this disclosure can occur in various orders and/or concurrently, and with other acts not presented and described in this disclosure. Furthermore, not all illustrated acts may be required to implement the methodologies in accordance with certain aspects of this disclosure. In addition, those skilled in the art will understand and appreciate that the methodologies could alternatively be represented as a series of interrelated states via a state diagram or events. Additionally, it should be appreciated that the methodologies disclosed in this disclosure are capable of being stored on an article of manufacture to facilitate transporting and transferring such methodologies to computing devices. The term article of manufacture, as used in this disclosure, is intended to encompass a computer program accessible from a computer-readable device or storage media.

What is claimed is:

1. A system, comprising:
a memory that stores computer executable components;
a processor that executes computer executable components stored in the memory, wherein the computer executable components comprise:
a prompt component configured to generate a prompt at a client device requesting information regarding one or more reactions experienced by a patient in association with usage of a medication by the patient;
a reception component configured to receive first information at the client device regarding the one or more reactions experienced by the patient in response to the generating the prompt; and
a reporting component configured to provide the first information to a server device in response to reception of the first information, wherein based on provision of the first information to the server device, the server device is configured to store the first information in a reaction log for the patient and provide one or more authorized users access to the reaction log.

2. The system of claim 1, wherein the prompt comprises one or more questions regarding the one or more reactions and wherein the one or more questions are determined based on the medication.

3. The system of claim 1, wherein the prompt comprises one or more questions regarding the one or more reactions and wherein the one or more questions are determined based on the medication and a medical health history of the patient.

4. The system of claim 1, wherein the prompt comprises one or more questions regarding the one or more reactions and wherein the one or more questions are determined based on second information regarding one or more second reactions to the medication previously reported as experienced by the patient.

5. The system of claim 1, wherein the prompt component is further configured to generate the prompt based on a current context associated with the patient or the client device.

6. The system of claim 1, wherein the prompt component is configured to regularly generate the prompt in accordance with a defined schedule.

7. The system of claim 1, wherein the prompt component is configured to generate the prompt in response to a determination that the first information has not been received in accordance with a defined schedule.

8. The system of claim 1, wherein the prompt component is configured to generate the prompt based on reception of physiological data about the patient captured by one or more medical monitoring devices, and wherein the prompt comprises one or more questions regarding the one or more reactions and wherein the one or more questions are determined based on the physiological data.

9. The system of claim 1, wherein the reception component is further configured to receive second information at the client device regarding the one or more reactions experienced by the patient, wherein the second information comprises physiological data about the patient captured by one or more medical monitoring device, and
wherein the reporting component is further configured to provide the second information to the server device in response to reception of the second information, wherein based on provision of the second information to the server device, the server device is configured to store the second information in a reaction log for the patient.

10. The system of claim 1, wherein the computer executable components further comprise:
a notification component configured to receive a notification from the server device in response to a determination, based on the first information, that the patient has experienced or is experiencing a clinically significant reaction.

11. The system of claim 10, wherein the clinically significant reaction comprise a side effect and wherein the notification comprises information instructing the patient regarding a action to perform to stop or minimize the side effect.

12. The system of claim 10, wherein the clinically significant reaction comprise a side effect and wherein the notification comprises information instructing the patient regarding a change to the patient's medication regimen determined based on the side effect.

13. A system, comprising:
a memory that stores computer executable components;
a processor that executes computer executable components stored in the memory, wherein the computer executable components comprise:
a reaction information reception component configured to receive first information regarding one or more first reactions experienced by a first patient in association with usage of a medication by the first patient;
a logging component configured to generate a reaction log for the first patient comprising the first information and provide one or more authorized users access to the reaction log; and
a patient evaluation component configured to evaluate the first information to determine whether the first information indicates the first patient is experiencing or has experienced one or more clinically significant reactions to the medication.

14. The system of claim 13, wherein the computer executable components further comprises:
an evaluation response component configured to initiate or execute a response in response to a determination that the first patient is experiencing or has experienced the one or more clinically signification reactions.

15. The system of claim 14, wherein the response comprises generating and sending a notification to the patient, the patient's clinician or another entity regarding the one or more clinically significant reactions.

16. The system of claim 14, wherein the response comprises changing the patient's medication regimen, including at least one of: revoking the patient's prescription for the medication, changing the patient's prescription dosage for the medication, or prescribing the patient a new medication.

17. The system of claim 13, wherein the reaction information reception component is further configured to receive second information regarding one or more second reactions experienced by second patients in association with usage of the medication by the second patients, and
wherein the logging component is further configured to generate historical reaction information for the medication comprising the first reaction information and the second reaction information and provide the one or more authorized users access to the historical reaction information for the medication.

18. The system of claim 17, wherein the computer executable components further comprise:
a medication evaluation component configured to evaluate the first information and the second information using machine learning to determine whether one or more characteristics associated with patient reactions to the medication deviate from labeling information developed for the medication relative to a threshold degree of deviation.

19. The system of claim 18, wherein the computer executable components further comprise:
a notification component configured to generate and send a notification to an entity responsible for regulating usage of the medication in response to a determination that the one or more characteristics associated with the patient reactions to the medication deviate from the labeling information developed for the medication relative to the threshold degree of deviation.

20. The system of claim 17, wherein the computer executable components further comprise:
a medication evaluation component configured to evaluate the first information and the second information using machine learning to determine optimal dosages for prescribing the medication based one or more patient characteristics.

21. A method comprising:
receiving, by a system comprising a processor, reaction information regarding one or more reactions experienced by a patient in association with usage of a medication by the patient;
generating, by the system, a reaction log for the patient comprising the information;
providing, by the system, one or more authorized users access to the reaction log; and
evaluating, by the system, the reaction information to determine whether the reaction information indicates the patient is experiencing or has experienced one or more clinically significant reactions to the medication.

22. The method of claim 21, wherein in response to a determination that the reaction information indicates the patient is experiencing or has experienced the one or more clinically significant reactions to the medication, the method further comprises:
generating, by the system, a notification regarding the one or more clinically significant reactions; and
sending, by the system, the notification to the patient, the patient's clinician or another entity.

23. The method of claim 22, wherein in response to a determination that the reaction information indicates the patient is experiencing or has experienced the one or more clinically significant reactions to the medication, the method further comprises:
issuing, by the system, an update to the patient's medication regimen, including at least one of: revoking the patient's prescription for the medication, changing the patient's prescription dosage for the medication, or prescribing the patient a new medication.
